(19) United States
(12) Patent Application Publication
(10) Pub. No.: US 2009/0196915 A1
(43) Pub. Date: Aug. 6, 2009

(54) COMPOSITION AND METHODS OF MAKING AND USING INFLUENZA PROTEINS

(76) Inventors: Gary VAN NEST, Martinez, CA (US); Brian D. LIVINGSTON, Martinez, CA (US); Georg ROTH, San Anselmo, CA (US); Deborah A. HIGGINS, Danville, CA (US)

Correspondence Address: MORRISON & FOERSTER LLP, 755 PAGE MILL RD, PALO ALTO, CA, 94304-1018, US

(21) Appl. No.: 12/196,015

(22) Filed: Aug. 21, 2008

Related U.S. Application Data
(60) Provisional application No. 60/957,157, filed on Aug. 21, 2007.
(60) Provisional application No. 61/081,640, filed on Jul. 17, 2008.

(51) Int. Cl. A61K 9/127; A61K 9/14; A61K 39/145

(52) U.S. Cl. 424/450; 424/489; 424/209.1

(57) ABSTRACT

The invention provides compositions of influenza proteins, such as matrix and nucleoprotein, that are presented to an individual's immune system as multimeric displays to induce an immune response. The compositions are optionally associated with any type of immunomodulatory compound (IMC) comprising an immunostimulatory sequences (ISS). The invention further provides compositions of influenza matrix and nucleoproteins that can induce cellular and/or humoral immune response. The invention also provides methods of making and using these compositions, e.g., as a vaccine, for ameliorating symptoms associated with infection with influenza virus or for reducing the risk of infection with influenza virus.

### FIELD OF THE INVENTION

This invention relates to the field of viruses, in particular influenza virus and compositions containing various influenza proteins. These compositions are useful for inducing immune responses against influenza, reducing the risk of infection from influenza, and/or ameloriating the symptoms of infection with influenza virus.

### BACKGROUND OF THE INVENTION

As set forth by the World Health Organization (WHO), influenza virus types A and B are both common causes of acute respiratory illnesses. Although both virus types may cause epidemics of considerable morbidity and mortality, influenza B infections are often limited to localized outbreaks, whereas influenza A viruses are the principal cause of larger epidemics, including worldwide pandemics. The influenza virus is a member of the Orthomyxovirus family, and has a wide individual range, including humans, horses, dogs, birds, and pigs. It is an enveloped, negative-sense RNA virus produced in 8 RNA segments encoding 10 viral proteins. The virus replicates in the nucleus of an infected individual cell. The influenza virus is most dangerous for the young and the old, or immunocompromised individuals. The virus can be propagated to high titers in chicken eggs, which serve as the vehicle for generation of virus for the production of influenza vaccines.

Two types of influenza vaccines are presently in use. The more conventional vaccine is an inactivated vaccine (containing killed virus) that is given by injection, typically into the arm. The most common human vaccine is the trivalent influenza vaccine (TIV) that contains purified and inactivated material from three viral strains. Typically this vaccine includes material from two influenza A virus subtypes and one influenza B virus strain. A second vaccine, called the nasal-spray flu vaccine (sometimes referred to as LAIV for Live Attenuated Influenza Vaccine), was approved in 2003 and contains attenuated (weakened) live viruses administered by nasal sprayer.

Influenza A viruses undergo frequent changes in their surface antigens, whereas type B influenza viruses change less frequently. Immunity following infection by one strain may not protect fully against subsequent antigenic variants. As a consequence, new vaccines against influenza must be designed each year to match the circulating strains that are most likely to cause the next epidemic. Therefore, the WHO annually collects data based on the surveillance of the most prevalent influenza strains circulating among people and makes recommendations for the influenza vaccine composition. Currently, the vaccine includes two subtypes of influenza A virus and one influenza B virus in the vaccine. The vaccine typically protects approximately 50%-80% of healthy adults against clinical disease.

Despite the availability of tie influenza vaccines , rates of illness among children, the elderly and certain high-risk groups is still significant, and in developing countries, vaccination may be sporadic or non-existent. In industrialized countries, production of sufficient influenza vaccine to accommodate the recipient population is hampered by production problems, high expenses and the time required to produce the vaccine using current technologies. In addition, threats of new viral strains and the possibility of future pandemics have raised interest in more effective and efficiently produced influenza vaccines.

Various groups have conducted research on some influenza proteins, such as matrix, to determine their immunogenicity and possible use as part of a vaccine against influenza. See, for example, Filette et al, Vaccine, 24:6597-601 (2006) and Liu et al., Vaccine, 23: 366-371 (2004). However, to date, there is a lack of a universal vaccine for influenza, especially one that induces humoral and cellular immune responses in an individual. Therefore, there is a need for improved influenza vaccines that provide long-lasting and effective protection against multiple strains of influenza virus.

### BRIEF SUMMARY OF THE INVENTION

The invention provides for compositions and vaccines comprising influenza proteins and methods of making and using them. In some embodiments, the compositions and vaccines additionally comprise an immunomodulatory compound (IMC) that comprises an immunostimulatory sequence (ISS).

In one aspect, the invention provides for compositions comprising a multimer of an extracellular domain of influenza matrix protein (M2e) which is presented to the immune system as a multimeric display and is capable of inducing an immune response in an individual. In some instances, the multimeric display is accomplished by association with a non-protein carrier. In one embodiment, the multimer comprises at least two copies of M2e. In another embodiment, the M2e multimer is associated with an IMC.

In other aspects, the M2e multimer or M2IMC multimers additionally comprise nucleoprotein (NP). In one embodiment, the multimer is a fusion protein comprising NP and M2e. In another embodiment, the M2e is covalently or ionically linked to NP. In some embodiments, the M2e is situated on the carboxy terminus side of NP. In other embodiments, the M2e is situated on the amino terminus side of NP. In other embodiments, the M2e is situated on both the amino terminus side and the carboxy terminus side of NP. In other embodiments, the M2e is situated internally to NP. In another embodiment, the M2e/IMC multimer is associated with NP. In another embodiment, the M2IMC multimer is associated with NP/IMC. In other embodiments, the M2e/NP multimer is associated with IMC. In some embodiments, the IMC is selected from the group consisting of 1018, type B oligonucleotides, chimeric immumodulatory compounds, and type C oligonucleotides.

In another aspect, the invention provides for any of the compositions above additionally comprising a carrier. In some embodiments, the carrier is selected from the group consisting of alum, microparticles, liposomes, and nanoparticles.

In another aspect, the invention provides for vaccines comprising a composition of a M2e multimer which is presented to the immune system as a multimeric display and is capable of inducing an immune response in an individual. In some embodiments, the composition further comprises an IMC, adjuvant or a carrier. In other embodiments, the composition further comprises NP. In other embodiments, the composition is a fusion protein comprising at least 2 copies of M2e and NP. In other embodiments, any of the compositions above further comprises IMC. In other embodiments, the vaccines further comprising a carrier selected from the group consisting of alum, microparticles, liposomes, and nanoparticles. In other embodiments, the vaccines comprise an IMC selected from the group consisting of 1018 IMC, type B oligonucleotides, chimeric immumodulatory compounds, and type C oligonucleotides. In another embodiment, any of the vaccines above further comprises one or more components of at least one trivalent inactivated influenza vaccine (TIV). In some embodiments, the TIV is selected from the group consisting of Fluzone, Fluvirin, Fluarix, FluLaval, FluBlok, FluAd, Influvac, and Fluvax.

In another aspect, the invention provides for methods for ameliorating one or more symptoms associated with infection with influenza virus in an individual by administering to the individual a vaccine comprising a multimer of an extracellular domain of influenza matrix protein (M2e) which is presented to the immune system as a multimeric display and wherein the multimer is capable of inducing an immune response in an individual. In one embodiment, the vaccine further comprises NP. In some embodiments, the vaccines further comprise an IMC.

In another aspect, the invention provides for methods for reducing the likelihood of infection with influenza virus in an individual comprising administering to the individual: (a) a vaccine comprising at least two copies of M2e and (b) one or more components of TIV. In one embodiment, the vaccine further comprises NP. In other embodiments, the vaccines further comprise an IMC. In other embodiments, the TIV is selected from the group consisting of Fluzone, Fluvirin, Fluarix, FluLaval, FluBlok, FluAd, Influvac, and Fluvax.

In another aspect, the invention provides for methods for reducing the likelihood of infection with influenza virus in an individual comprising administering to the individual: (a) a vaccine comprising at least two copies of M2e and (b) one or more components of monovalent inactivated vaccine.

### BRIEF DESCRIPTION OF THE DRAWINGS

FIG. 1 depicts a consensus M2e sequences for human, swine and avian species and the conservation of M2e epitopes among various influenza A isolates. The variants of the consensus sequences are also shown for different strains of influenza virus.

FIG. 2 depicts a comparison to the 1990-2005 consensus NP sequence with the NP sequence of A/Puerto Rico/8/34 (H1N1). Based on amino acid similarity matrixes, conservative changes are highlighted as indicated in dashed boxes, neutral are single line boxes and non-conservative are double line boxes.

### DETAILED DESCRIPTION OF THE INVENTION

The invention provides for compositions and/or vaccines comprising influenza proteins and methods for making and using them. These compositions and vaccines are useful for inducing immune responses in individuals infected with influenza virus. Additionally, the compositions and vaccines are useful for ameliorating symptoms associated with infection with influenza virus and reducing the risk of infection with influenza virus.

### General Methods

The practice of the present invention will employ, unless otherwise indicated, conventional techniques of molecular biology (including recombinant techniques), microbiology, cell biology, biochemistry, nucleic acid chemistry, and immunology, which are within the skill of the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly and individually referred to herein as “Sambrook”). Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987, including supplements through 2001); PCR: The Polymerase Chain Reaction, (Mullis et al., eds., 1994); Current Protocols in Immunology (J. E. Coligan et al., eds., 1991); The Immunoassay Handbook (D. Wild, ed., Stockton Press N.Y., 1994); Bioconjugate Techniques (Greg T. Hermanson, ed., Academic Press, 1996); Methods of Immunological Analysis (R. Masseyeff, W. H. Albert, and N. A. Staines, eds., Weinheim: VCH Verlags gesellsclaft mbH, 1993), Harlow and Lane (1988) Antibodies, A Laboratory Manual, Cold Spring Harbor Publications, New York, and Harlow and Lane (1999) Using Antibodies: A Laboratory Manual Cold Spring Harbor Laboratory Press, Cold Spring Harbor, N.Y. (jointly and individually referred to herein as “Harlow and Lane”), Beaucage et al. eds., Current Protocols in Nucleic Acid Chemistry John Wiley & Sons, Inc., New York, 2000); and Agrawal, ed., Protocols for Oligonucleotides and Analogs, Synthesis and Properties Humana Press Inc., New Jersey, 1993).

### Definitions

As used herein, a “vaccine” is an antigenic preparation that is used to induce an immune response in individuals. A vaccine can more have than one constituent that is antigenic.

As used herein, “multimeric display” refers to the way that a molecule, such as matrix (M2e), is presented. In one embodiment, this refers to the way the molecule is displayed to an individual's immune system. Multimeric display includes but is not limited to, association with polymers, or repeating units of the molecule displayed linearly (e.g., end-to-end) with or without spacer regions, and multiple units of the molecule displayed in a non-linear manner (e.g., radial display, random orientation of the molecules, etc.). The multiple units can be displayed physically by association with a carrier or any type of platform molecule, including but not limited to, other influenza proteins (e.g., nucleoprotein), non-influenza proteins or non-protein platform molecules such as microcarriers, aluminum salts, other inorganic salts, microparticles, nanoparticles, virus-like particles, dendromers, micelles, natural or synthetic polymers and liposomes.

As used herein, “non-protein carriers” are carriers which are not proteins and can be used to achieve multimeric display of influenza matrix and/or nucleoprotein.

As used interchangeably herein, the terms “polynucleotide,” “oligonucleotide” and “nucleic acid” include single-stranded DNA (ssDNA), double-stranded DNA (dsDNA), single-stranded RNA (ssRNA) and double-stranded RNA (dsRNA), modified oligonucleotides and oligonucleosides, or combinations thereof. The nucleic acid can be linearly or circularly configured, or the oligonucleotide can contain both linear and circular segments. Nucleic acids are polymers of nucleosides joined, e.g., through phosphodiester linkages or alternate linkages, such as phosphorothioate esters. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, deoxythymidine, and deoxycytidine. A nucleotide is a phosphate ester of a nucleoside.

The term “ISS” or “immunostimulatory sequence” as used herein refers to polynucleotide sequences that effect a measurable immune response as measured in vitro, in vivo and/or ex vivo. Examples of measurable immune responses include, but are not limited to, antigen-specific antibody production, secretion of cytokines, activation or expansion of lymphocyte populations such as NK cells, CD4+ T lymphocytes, CD8+ T lymphocytes, B lymphocytes, and the like. Preferably, the ISS sequences preferentially activate a Th1-type response. A polynucleotide for use in the invention contains at least one ISS. As used herein, “ISS” is also a shorthand term for an ISS-containing polynucleotide.

The term “immunomodulatory compound” or “IMC”, as used herein, refers to a molecule which has immunomodulatory activity and which comprises a nucleic acid moiety comprising an immunostimulatory sequence or ISS. The IMC may consist of a nucleic acid moiety that comprises more than one ISS, consists of an ISS, or has no immunomodulatory activity on its own. The IMC may consist of an oligonucleotide (an “oligonucleotide IMC”) or it may comprise additional moieties. Accordingly, the term IMC includes chimeric immunomodulatory compounds (“CICs”) which incorporate two or more nucleic acid moieties, at least one of which comprises the sequence 5′-CG-3′, covalently linked to a non-nucleotide spacer moiety.

The term “immunomodulatory” can refer to the particulate composition and/or the polynucleotide. Thus, an immunomodulatory composition of the invention may exhibit immunomodulatory activity even when the polynucleotide contained in the composition has a sequence that, if presented as a polynucleotide alone, does not exhibit comparable immunomodulatory activity. In some embodiments, when presented alone, a polynucleotide of an immunomodulatory composition of the invention does not have “isolated immunomodulatory activity,” or has “inferior isolated immunomodulatory activity,” (i.e., when compared to particulate composition). The “isolated immunomodulatory activity” of a polynucleotide is determined by measuring the immunomodulatory activity of the isolated polynucleotide having the same nucleic acid backbone (e.g., phosphorothioate, phosphodiester, chimeric) using standard assays which indicate at least one aspect of an immune response, such as those described herein.

The term “conjugate” refers to a complex in which an IMC and a multimer are linked. Such conjugate linkages include covalent and/or non-covalent linkages.

The term “associated with” can refer to both covalent as well as non-covalent interactions. For example, an M2e can be associated with an IMC by covalent linkage to the IMC as well as non-covalent interactions with the IMC.

“Adjuvant” refers to a substance which, when added to an immunogenic agent such as antigen, nonspecifically enhances or potentiates an immune response to the agent in the recipient individual upon exposure to the mixture.

The term “microcarrier” refers to a particulate composition which is insoluble in water and which has a size of less than about 150, 120 or 100 μm, more commonly less than about 50-60 μm, and may be less than about 10 μm or even less than about 5 μm. Microcarriers include “nanocarriers,” which are microcarriers have a size of less than about 1 μm, preferably less than about 500 nm. Microcarriers include solid phase particles such particles formed from biocompatible naturally occurring polymers, synthetic polymers or synthetic copolymers, although microcarriers formed from agarose or cross-linked agarose may be included or excluded from the definition of microcarriers herein as well as other biodegradable materials known in the art. Solid phase microcarriers are formed from polymers or other materials which are non-erodible and/or non-degradable under mammalian physiological conditions, such as polystyrene, polypropylene, silica, ceramic, polyacrylamide, gold, latex, hydroxyapatite, and ferromagnetic and paramagnetic materials. Biodegradable solid phase microcarriers may be formed from polymers which are degradable (e.g., poly(lactic acid), poly(glycolic acid) and copolymers thereof, such as poly(D,L-lactide-co-glycolide) or erodible (e.g., poly(ortho esters such as 3,9-diethylidene-2,4,8,10-tetraoxaspiro[5.5] undecane (DETOSU) or poly(anhydrides), such as poly(anhydrides) of sebacic acid) under mammalian physiological conditions. Microcarriers are typically spherical in shape, but microcarriers which deviate from spherical shape are also acceptable (e.g., ellipsoidal, rod-shaped, etc.). Due to their insoluble nature, some solid phase microcarriers are filterable from water and water-based (aqueous) solutions (e.g., using a 0.2 micron filter). Microcarriers may also be liquid phase (e.g., oil or lipid based), such as liposomes, iscoms (immune-stimulating complexes, which are stable complexes of cholesterol, phospholipid and adjuvant-active saponin) without antigen, or droplets or micelles found in oil-in-water or water-in-oil emulsions, such as MF59. Biodegradable liquid phase microcarriers typically incorporate a biodegradable oil, a number of which are known in the art, including squalene and vegetable oils. The term “nonbiodegradable”, as used herein, refers to a microcarrier which is not degraded or eroded under normal mammalian physiological conditions. Generally, a microcarrier is considered nonbiodegradable if it not degraded (i.e., loses less than 5% of its mass or average polymer length) after a 72 hour incubation at 37° C. in normal human serum.

An “individual” or “subject” is a vertebrate, such as avian, preferably a mammal, such as a human. Mammals include, but are not limited to, humans, non-human primates, farm animals, sport animals, experimental animals, rodents (e.g., mice and rats) and pets.

An “effective amount” or a “sufficient amount” of a substance is that amount sufficient to effect a desired biological effect, such as beneficial results, including clinical results, and, as such, an “effective amount” depends upon the context in which it is being applied. In the context of this invention, an example of an effective amount of a composition comprising a multimer of an extracellular domain of influenza matrix protein (M2e) is an amount sufficient to induce an immune response in an individual. An effective amount can be administered in one or more administrations.

The tern “co-administration” as used herein refers to the administration of at least two different substances sufficiently close in time to modulate an immune response. Preferably, co-administration refers to simultaneous administration of at least two different substances.

“Stimulation” of an immune response, such as humoral or cellular immune response, means an increase in the response, which can arise from eliciting and/or enhancement of a response.

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of infection, stabilized (i.e., not worsening) state of infection, amelioration or palliation of the infectious state, and remission (whether partial or total), whether detectable or undetectable. “Treatment” can also mean prolonging survival as compared to expected survival if not receiving treatment.

### Compositions of Influenza Proteins

The matrix proteins M1 and M2 are encoded by genome 7 of the influenza A virus. The extracellular portion of this influenza A M2-protein is also known as M2e and is 23 amino acids long. It is minimally immunogenic during infection and conventional vaccination and has high sequence conservation across all human influenza A strains. One advantage of M2e as an antigen is the conservation of its sequence that has hardly changed since the first influenza virus was isolated in 1933, despite numerous epidemics and several pandemics.

The invention provides for compositions comprising a multimer of an extracellular domain of influenza matrix protein (M2e) wherein the multimer is capable of inducing an immune response in an individual. In one aspect, the multimer of M2e protein comprises at least two copies of M2e. Without being bound by theory, multiple copies of M2e are important for inducing an immune response in an individual because the multiple copies of M2e allow for the M2e to be presented to an individual's immune system as a multimeric display. Accordingly, in one embodiment, the composition comprises two copies of M2e. In other embodiments, the composition comprises 3, 4, or 5 copies of M2e. In yet other embodiments, the composition comprises 6, 7, or 8 copies of M2e. In yet other embodiments, the composition comprises 9, 10, 11 or 12 copies of M2e. In yet other embodiments, the composition comprises more than 12 copies of M2e. The M2e multimers may also be linked to an IMC comprising animmunostimulatory sequence (IMC), as described in greater detail herein. Multimers may be made by any method known to one of skill in the art, including but not limited to, the use of platform molecules. The Examples illustrate a few embodiments of how one of skill in the art can make and use multimers of the invention.

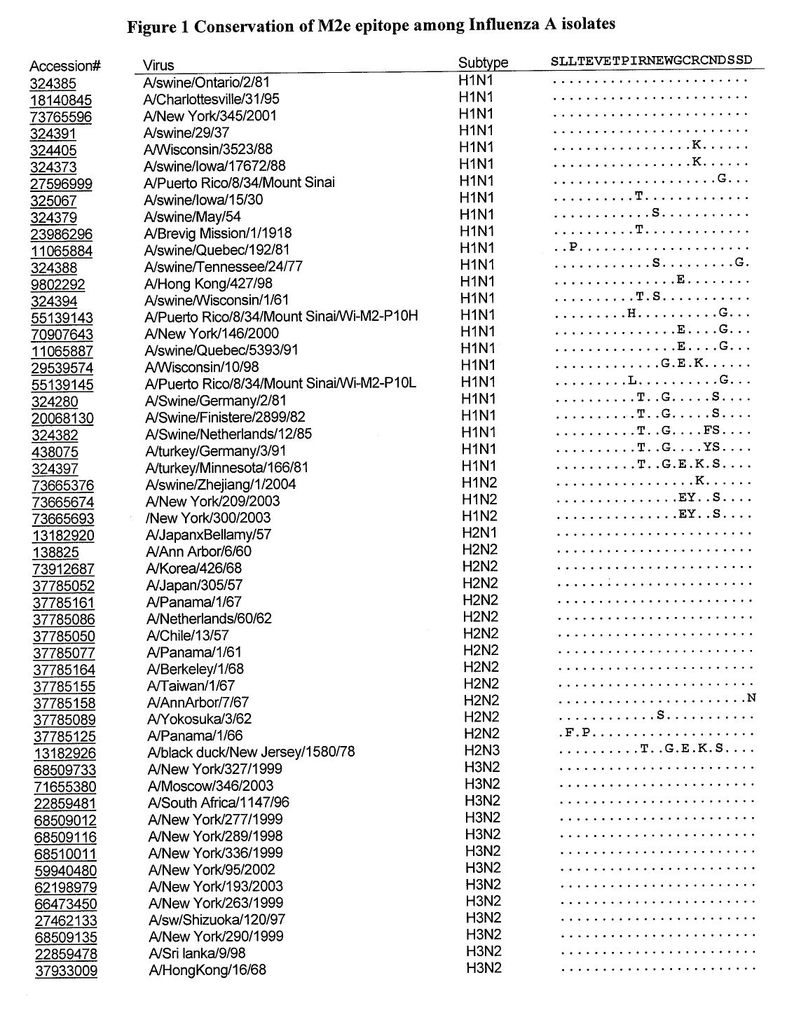

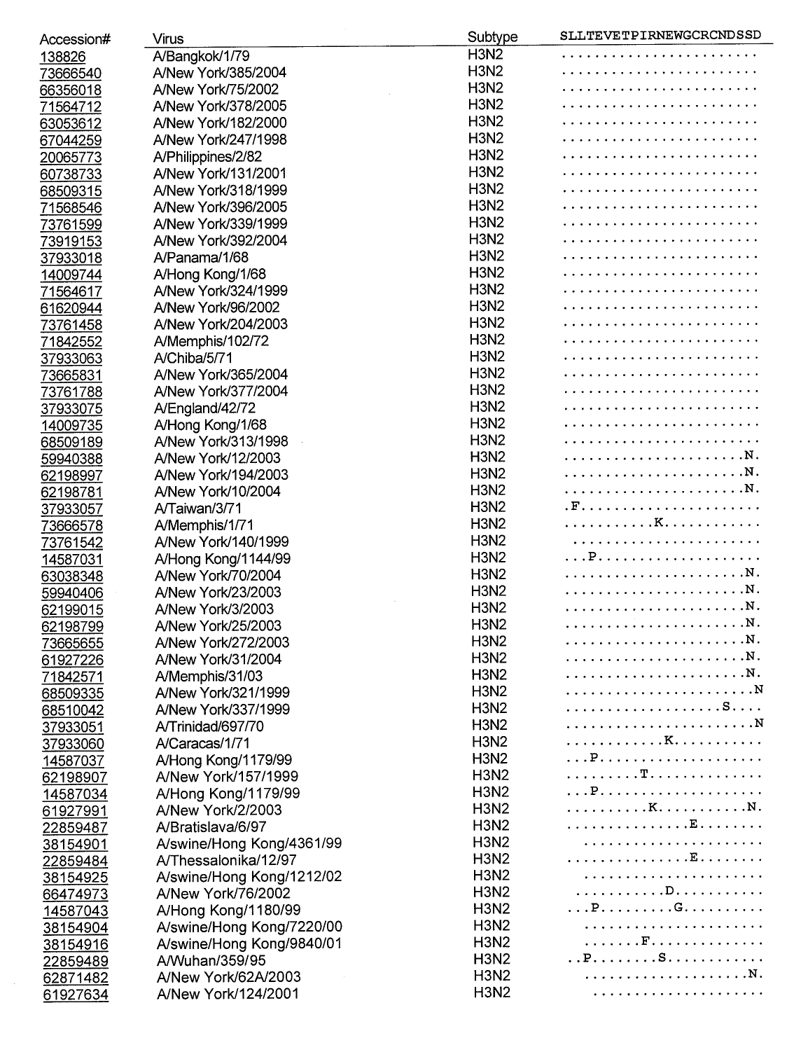

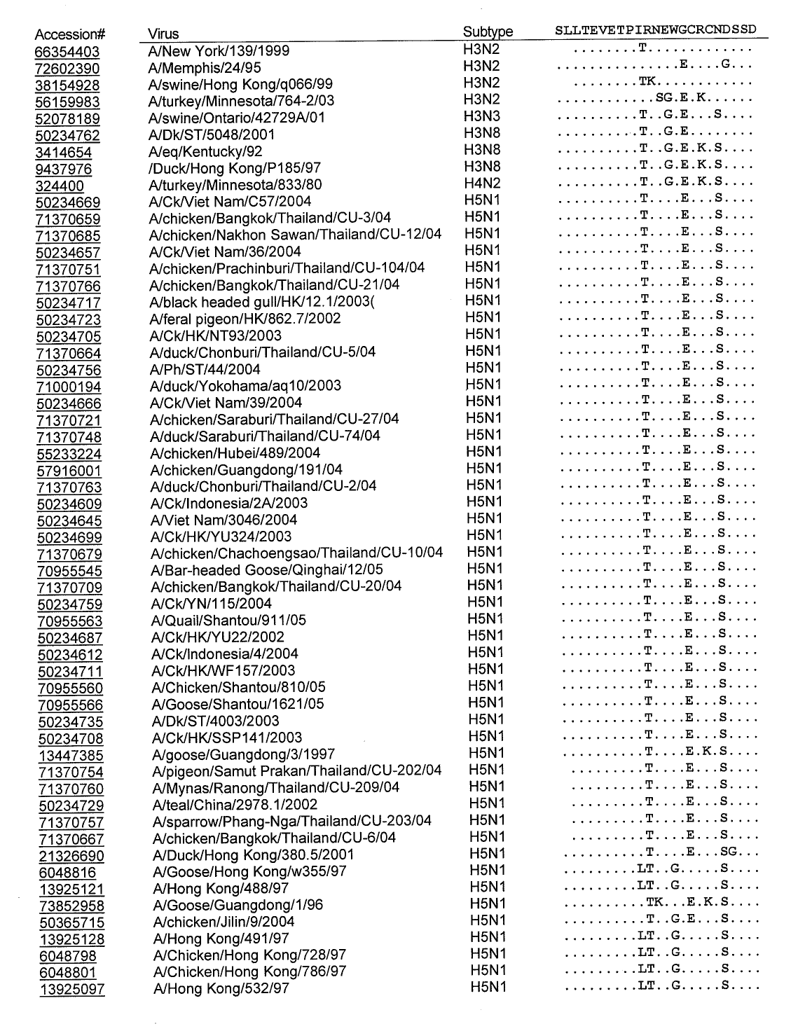

The invention also provides for compositions comprising a multimer of M2c of various sequences. The multimer may include M2e copies of the same sequence or of varying sequences. The consensus sequence of human M2e is SLLTEVETPIRNEWGCRCNDSSD (SEQ ID NO: 7). The consensus sequence for swine M2e is SLLTEVETPIRNGWECRCNDSSD (SEQ ID NO: 8). The consensus sequence of avian M2e is SLLTEVETPTRNGWECKCSDSSD (SEQ ID NO: 9). FIG. 1 shows this consensus sequence as well as the consensus sequences for swine and avian animals. However, as FIG. 1 depicts, there are a number of isolates within influenza A and in some of the isolates, there are one or more amino acid variations from the consensus sequence. The invention contemplates the use of the combination of any of these isolates to generate a multimer of M2e (optionally with an IMC) in a composition. The composition can then be formulated for use as a vaccine and/or in a suitable form for administration to an individual as described herein. In particular, the composition can comprise M2e proteins with sequences that are from isolates of great public health interest. In one aspect, the invention provides for compositions comprising multimers of M2e from the H5N1 strain to induce immune response in individuals in need thereof. These compositions may be used prophylactically to reduce the likelihood of infection with avian influenza virus or to treat symptoms associated with infection with avian influenza virus.

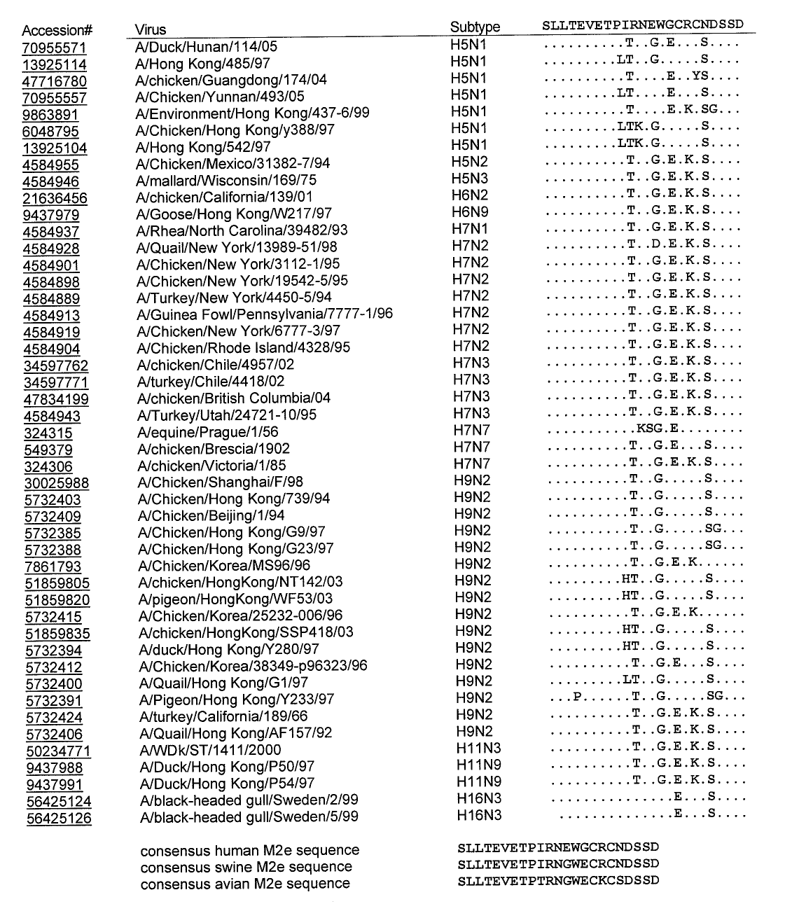

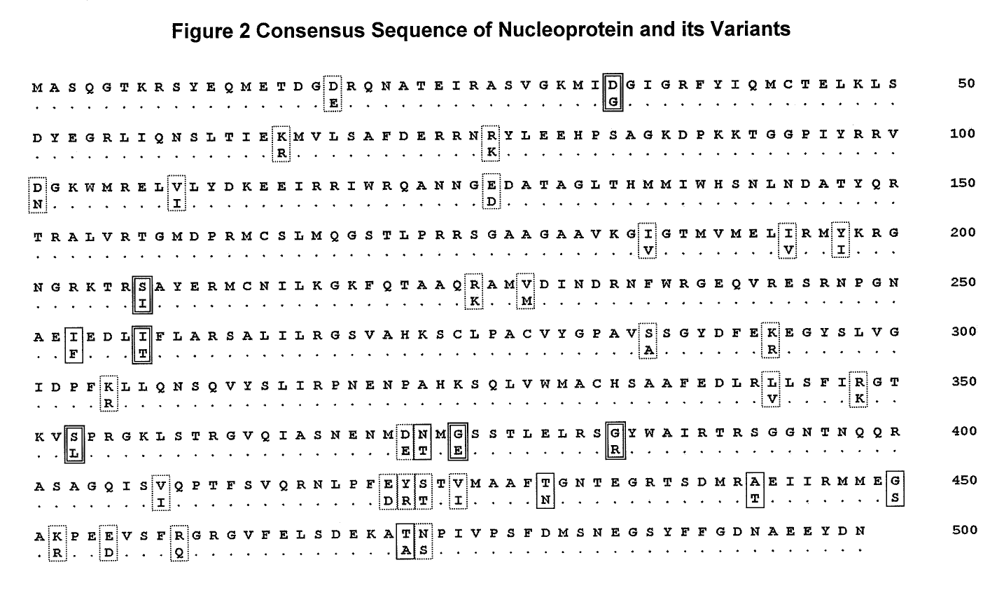

In other aspects of the invention, the composition comprises one or more multimers of M2e and nucleoprotein (NP). FIG. 2 shows the consensus sequence of nucleoprotein with its variants. Of the 815 full length human influenza NP sequences present in GenBank, 76% are derived from viruses isolated between the years of 1990-2005. In this time period, 82% (503 sequences) are from H3N2 isolates. A consensus NP sequence was generated based on all full length human NP sequences from 1990-2005 isolates (FIG. 2). A comparison of the A/Puerto Rico/8/34 (H1N1) sequence against the post 1990 consensus sequence found there is 92% amino acid sequence identity. The A/Puerto Rico/8/34 (H1N1) NP sequence has 98% sequence similarity to the consensus. Based on a Blosum 45 amino acid similarity matrix, 12 or the amino acid differences were found to be nonconservative or neutral substitutions. The consensus H3N2 sequence bears three unique amino acid substitutions at positions 98, 146 and 197, in each case the substitution is conserved. It is contemplated that NP may be expressed with a single copy or in multiple copies. In one embodiment, NP is expressed as dimer. In another embodiment, the NP associates into a higher order structures, such as a trimer.

In another aspect, the M2e copies and NP are expressed as a fusion protein. The M2e polynucleotide sequences can be cloned into any suitable expression vector and used to express a protein sequence that is desired for the composition. The Examples disclose both the polynucleotide and protein sequence of fusion protein constructs with M2e and NP that can be used in practicing this aspect of the invention. The composition can also comprise M2e and NP in a manner that is not a fusion protein, for example, as associated with each via covalent linkage, ionic linkage or by other physical forces (e.g., Van de Waals).

The invention also provides for compositions and fusion proteins which comprise one or more multimers of M2e and nucleoprotein (NP) in different orientations. These fusion proteins may additionally comprise one or more histidine residues (“his tags”), preferably six histine residues, at their carboxy terminus. In one aspect, one or more M2e proteins are situated on the amino terminus side of NP. In another aspect, one or more M2e proteins are situated on the carboxy terminus side of NP. In another aspect, one or more M2e proteins are situated on both the amino terminus and the carboxy terminus side of NP. In other aspects, the M2e is situated internally within the NP sequence(s). In yet other aspects, M2e and NP alternate with each other. In particularly preferred embodiments, 4 or 8 copies of the M2e protein are situated on the amino or carboxy termini of NP. In one particularly preferred embodiment, 4 copies of the M2e protein are situated on both the amino and carboxy termini of NP. In all embodiments, spacer sequences may optionally be included after one or more copies of the M2e protein.

Without being bound by theory, the use of the NP can assist in the induction of the cytotoxic T lymphocyte (CTL) and interferon (e.g., IFN-γ) responses that may contribute to the control of influenza infection. The M2e can assist in the induction of antibody responses against the influenza virus. The CTL response and the antibody response can work synergistically to augment an individual's immune to a greater extent than either one alone. Furthermore, the NP may also provide helper T lymphocyte epitopes that may result in augmenting M2e antibody responses.

The compositions of the invention, either multimeric M2c or multimeric M2c/NP can additionally comprise an immunomodulatory compound comprising an immunostimulatory sequence (IMC), which are described in greater detail below. In a preferred embodiment, the multimers are expressed as a fusion protein. The multimers optionally are associated with an IMC. One advantage of expressing the M2e and NP as a fusion protein and conjugating the fusion protein to the IMC is easier production. Instead of expressing each influenza protein as a separate protein and separately conjugating them, both proteins are expressed at one time and conjugated to the IMC, thereby simplifying the production process.

### Immunomodulatory Compounds (IMCs) and Immunostimulatory Sequences (ISS)

The compositions and methods of this invention can be utilized with any type of immunomodulatory compound (IMC) comprising an immunostimulatory sequence (IMC). The term “IMC” as used herein refers to oligonucleotide sequences that effect a measurable immune response as measured in vitro, in vivo and/or ex vivo. IMC contain an unmethylated cytosine, guanine dinucleotide sequence (e.g., “CpG” or DNA containing a cytosine followed by guanosine and linked by a phosphate bond) and stimulates the immune system. Various methods for determining the stimulation of the immune system are described below. Immunostimulatory sequences and/or immunostimulatory nucleic acids have been described in the art. For example, the immunostimulatory nucleic acids have been described in U.S. Pat. Nos. 6,194,388; 6,207,646; and 6,239,116. IMC have been described in various publications. See, for example, U.S. Publication No. 20060058254; WO 2004/058179; U.S. Pat. No. 6,589,940; U.S. Publication No. 20040006034; U.S. Publication No. 20070027098; WO 98/55495. In addition, the class of immunostimulatory nucleic acids known as chimeric immunomodulatory compounds (CICs) can also be used with the multimers of the invention. See, for example, U.S. Pat. No. 7;255,868; U.S. Publication No. 20030199466; U.S. Publication No. 20070049550; U.S. Publication No. 20030225016; U.S. Publication No. 20040132677 and WO 03/000922.

IMC in general can be of any length greater than 8 bases or base pairs. In other embodiments, the IMC is at least 10, 15, or 20 bases or base pairs in length. In some embodiments, the IMC is at most 30, 50, 60, 80 or 100 bases or base pairs in length. The IMC contains a CpG motif represented by the formula: 5′-X₁X₂CGX₃X₄-3′, wherein X₁, X₂, X₃ and X₄ are nucleotides. In one aspect, the IMC of the invention can include a) a palindromic sequence at least 8 bases in length which contains at least one CG dinucleotide and b) at least one TCG trinucleotide at or near the 5′ end of the polynucleotide. The IMC contains at least one palindromic sequence of at least 8 bases in length containing at least one CG dinucleotide. The IMC can also contain at least one TCG trinucleotide sequence at or near the 5′end of the polynucleotide (i.e., 5′-TCG). In some instances, the palindromic sequence and the 5′-TCG are separated by 0, 1 or 2 bases in the IMC. In some instances the palindromic sequence includes all or part of the 5′-TCG. These IMC are more fully described in U.S. Publication No. 20060058254 and WO 2004/058179.

In another aspect, the IMC of the invention comprise octameric IMCs, which comprise a CG containing sequence of the general octameric sequence 5′-Purine, Purine, Cytosine, Guanine, Pyrimidine, Pyrimidine, Cytosine, (Cytosine or Guanine)-3′. As is readily evident to one skilled in the art, this class of sequences encompasses the following: GACGTTCC; GACGCTCC; GACGTCCC; GACGCCCC; AGCGTTCC; AGCGCTCC; AGCGTCCC; AGCGCCCC; AACGTTCC; AACGCTCC; AACGTCCC; AACGCCCC; GGCGTTCC; GGCGCTCC; GGCGTCCC; GGCGCCCC; GACGTTCG; GACGCTCG; GACGTCCG; GACGCCCG; AGCGTTCG; AGCGCTCG; AGCGTCCG; AGCGCCCG; AACGTTCG; AACGCTCG; AACGTCCG; AACGCCCG; GGCGTTCG; GGCGCTCG; GGCGTCCG; GGCGCCCG. The IMC can also comprise an octamer selected from the group consisting of: AACGTTCC, AACGTTCG, GACGTTCC, and GACGTTCG. In one embodiment, the IMC octamer comprises 5′-purine, purine, cytosine, guanine, pyrimidine, pyrimidine, cytosine, guanine-3′ or the IMC octamer comprises 5′-purine, purine, cytosine, guanine, pyrimidine, pyrimidine, cytosine, cytosine-3′. The IMC octanucleotide can also comprise 5′-GACGTTCG-3′, 5′-GACGTTCC-3′, 5′-AACGTTCG-3′ or 5′-AACGTTCC-3′.

In another aspect, an IMC comprising or consisting of the 1018 IMC can be used in association (covalent or non-covalent) with the M2e or M2e/NP multimers of the invention. The structure of 1018 IMC has been published in multiple scientific articles as well as patents. See, for example, Hessel et al. (2005) J. Exp. Med., 202(11):1563. In general, 1018 IMC is (5′-TGACTGTGAACGTTCGAGATGA -3′)(SEQ ID NO: 10).

IMCs such as chimeric immunomodulatory compounds (“CICs”) can also be used with the M2e or M2e/NP multimers of the invention. CICs generally comprise one or more nucleic acid moieties and one or more non-nucleic acid moieties. The nucleic acid moieties in a CIC with more than one nucleic acid moiety may be the same or different. The non-nucleic acid moieties in a CIC with more than one non-nucleic acid moiety may be the same or different. Thus, in one embodiment the CIC comprises two or more nucleic acid moieties and one or more non-nucleic acid spacer moieties, where at least one non-nucleic acid spacer moiety is covalently joined to two nucleic acid moieties. In an embodiment, at least one nucleic acid moiety comprises the sequence 5′-CG-3′. In an embodiment, at least one nucleic acid moiety comprises the sequence 5′-TCG-3′.

Delivery of M2e or M2e/NP Multimers

In one embodiment, the M2e or M2e/NP multimer is delivered by itself into the individual. In another embodiment, the multimers are delivered with one or more IMC. In one embodiment, the multimer is co-administered with the IMC as a conjugate. In another embodiment, the multimer is administered with the IMC in a separate vehicle. The administration of the multimer can be contemporaneous or simultaneous with the IMC. Discussion of delivery of the IMC infra also contemplates delivery of the multimer with the IMC.

The influenza multimers and/or multimer/IMC can also be administered with other influenza vaccines to enhance the efficacy of the influenza vaccines. Types of influenza vaccines which are contemplated for use with the influenza multimers and/or multimer/IMC include but are not limited to whole virus vaccines, split virus vaccines, subunit purified virus vaccines, recombinant subunit vaccines and recombinant virus vaccines.

Additionally, the multimers or multimer/IMC may also be delivered with one or more components of multivalent vaccines for influenza (e.g., monovalent, divalent, or trivalent). In one aspect, compositions of multimers or multimer/IMC are delivered with one or more components of trivalent inactivated vaccines (TIV) for influenza. The standard components of TIV include hemagglutinin (HA) and neuraminidase from three different strains of influenza virus. Examples of TIV which may be used include, but are not limited to, Fluzone, Fluvirin, Fluarix, FluLaval, FluBlok, FluAd, Influvac, and Fluvax. The TIVs are used in the amounts that have been approved for use by the Food and Drug Administration (FDA). Divalent influenza vaccines (DIV) would contain hemagglutinin from two different influenza strains. Monovalent influenza vaccines (MIV) would contain hemagglutinin and neurminidase from only one influenza strain such as H5N1. TIV, DIV, and MIV could also contain only hemagglutinin from three, two, or one influenza strains without containing the neuraminidase component. Additionally, the multimers or multimer/IMC may also be delivered with influenza vaccines containing hemagglutinin and neuraminidase from more than three separated influenza strains (quadravalent or higher). These TIV, DIV, and MIV can be administered contemporaneously with the multimer or multimer/IMC compositions or at intervals before or after the administration of multimer or multimer/IMC compositions. In one aspect, the multimers or multimer/IMC may be administered to an individual before the administration of TIV, DIV, or MIV to enhance the response to the hemagglutinin-containing vaccine. In one embodiment, the multimers or multimer/IMC are administered about 1 day before the TIV, DIV, or MIV. In other embodiments, the multimers or multimer/IMC are administered about 2, 3, 4, 5, or 6 days before the TIV, DIV, or MIV. In other embodiments, the multimers or multimer/IMC are administered about 1 week before the TIV, DIV, or MIV. In other embodiments, the multimers or multimer/IMC are administered about 1.5 or 2 weeks before the TIV, DIV, or MIV. In other embodiments, the multimers or multimer/IMC are administered about 2.5, 3, 3.5, or 4 weeks before the TIV, DIV, or MIV.

The multimers or multimer/IMC may also be administered with a monovalent inactivated vaccine (MIV), such as that for the H5N1 strain. MIV contain hemagglutinin and neuraminidase from only one influenza strain. These MIV can be administered contemporaneously with the multimer or multimer/IMC compositions or at intervals before or after the administration of multimer or multimer/IMC compositions. In one aspect, the multimers or multimer/IMC may be administered to an individual before the administration of MIV to enhance the MIV response. In one embodiment, the multimers or multimer/IMC are administered about 1 day before the MIV. In other embodiments, the multimers or multimer/IMC are administered about 2, 3, 4, 5, or 6 days before the MIV. In other embodiments, the multimers or multimer/IMC are administered about 1 week before the MIV. In other embodiments, the multimers or multimer/IMC are administered about 1.5 or 2 weeks before the MIV. In other embodiments, the multimers or multimer/IMC are administered about 2.5, 3, 3.5, or 4 weeks before the MIV.

M2e, M2e/NP, M2e/IMC, and M2e/NP/IMC constructs may be incorporated into a delivery vector, such as a plasmid, cosmid, virus or retrovirus, which may in turn code for therapeutically beneficial polypeptides, such as cytokines, hormones and antigens. Incorporation of an IMC into such a vector does not adversely affect their activity.

A colloidal dispersion system may be used for targeted delivery of the compositions to an inflamed tissue, such as nasal membranes. Colloidal dispersion systems include macromolecule complexes, nanocapsules, microspheres, beads, and lipid-based systems including oil-in-water emulsions, micelles, mixed micelles, and liposomes. In one embodiment, the colloidal system of this invention is a liposome.

Liposomes are artificial membrane vesicles which are useful as delivery vehicles in vitro and in vivo. It has been shown that large unilamellar vesicles (LUV), which range in size from 0.2-4.0, um can encapsulate a substantial percentage of an aqueous buffer containing large macromolecules. RNA, DNA and intact virions can be encapsulated within the aqueous interior and be delivered to cells in a biologically active form (Fraley, et al, Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes encoding the antisense polynucleotides at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988).

The composition of the liposome is usually a combination of phospholipids, particularly high-phase transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various well known linking groups can be used for joining the lipid chains to the targeting ligand (see, e.g., Yanagawa, et al., Nuc. Acids Symp. Ser., 19:189 (1988); Grabarek, et al., Anal. Biochem., 185:131 (1990); Staros, et al., Anal. Biochem. 156:220 (1986) and Boujrad, et al., Proc. Natl. Acad. Sci. USA, 90:5728 (1993). Targeted delivery of multimers or multimer/IMC can also be achieved by conjugation of the IMC to the surface of viral and non-viral recombinant expression vectors, to an antigen or other ligand, to a monoclonal antibody or to any molecule which has the desired binding specificity.

Those of ordinary skill in the art will also be familiar with, or can readily determine, methods useful in preparing oligonucleotide-peptide conjugates. Conjugation can be accomplished at either terminus of an IMC oligonucleotide or at a suitably modified base in an internal position (e.g., a cytosine or uracil). For reference, methods for conjugating oligonucleotides to proteins and to oligosaccharide moieties of Ig are known (see, e.g., O'Shannessy, et al., J. Applied Biochem., 7:347 (1985). Another useful reference is Kessler: “Nonradioactive Labeling Methods for Nucleic Acids”, in Kricka (ed.), Nonisotopic DNA Probe Techniques (Acad. Press, 1992)).

Co-administration of a peptide drug with an oligonucleotide IMC according to the invention may also be achieved by incorporating the IMC in cis or in trans into a recombinant expression vector (plasmid, cosmid, virus or retrovirus) which codes for any therapeutically beneficial protein deliverable by a recombinant expression vector. If incorporation of an oligonucleotide IMC into an expression vector for use in practicing the invention is desired, such incorporation may be accomplished using conventional techniques which do not require detailed explanation to one of ordinary skill in the art. For review, however, those of ordinary skill may wish to consult Ausubel, Current Protocols in Molecular Biology, supra.

Briefly, construction of recombinant expression vectors (including those which do not code for any protein and are used as carriers for an oligonucleotide IMC) employs standard ligation techniques. For analysis to confirm correct sequences in vectors constructed, the ligation mixtures may be used to transform a individual cell and successful transformants selected by antibiotic resistance where appropriate. Vectors from the transformants are prepared, analyzed by restriction and/or sequenced by, for example, the method of Messing, et al., (Nucleic Acids Res., 9:309, 1981), the method of Maxam, et al., (Methods in Enzymology, 65:499, 1980), or other suitable methods which will be known to those skilled in the art. Size separation of cleaved fragments is performed using conventional gel electrophoresis as described, for example, by Maniatis, et al., (Molecular Cloning, pp. 133-134, 1982).

Individual cells may be transformed with expression vectors and cultured in conventional nutrient media modified as is appropriate for inducing promoters, selecting transformants or amplifying genes. The culture conditions, such as temperature, pH and the like, are those previously used with the individual cell selected for expression, and will be apparent to the ordinarily skilled artisan.

If a recombinant expression vector is utilized as a carrier for the oligonucleotide IMC used in the invention, plasmids and cosmids are particularly preferred for their lack of pathogenicity. However, plasmids and cosmids are subject to degradation in vivo more quickly than viruses and therefore may not deliver an adequate dosage of IMC to substantially inhibit ISS immunostimulatory activity exerted by a systemically administered gene therapy vector. Of the viral vector alternatives, adenoassociated viruses would possess the advantage of low pathogenicity. The relatively low capacity of adeno-associated viruses for insertion of foreign genes would pose no problem in this context due to the relatively small size in which oligonucleotide IMC of the invention can be synthesized. In one embodiment, a DNA vaccine or a viral vector is used to express the M2e multimers or M2e/NP multimers (optionally including an oligonucleotide IMC).

Other viral vectors that can be utilized in the invention include adenovirus, adeno-associated virus, herpes virus, vaccinia or an RNA virus such as a retrovirus. Retroviral vectors are preferably derivatives of a murine, avian or human HIV retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence that enables the packaging mechanism to recognize an RNA transcript for encapsidation. Helper cell lines that have deletions of the packaging signal include, but are not limited to, T2, PA317 and PA 12, for example. These cell lines produce empty virions, since no genome is packaged. If a retroviral vector is introduced into such helper cells in which the packaging signal is intact, but the structural genes are replaced by other genes of interest, the vector can be packaged and vector virion can be produced. By inserting one or more sequences of interest into the viral vector, along with another gene which encodes the ligand for a receptor on a specific target cell, for example, the vector can be rendered target specific. Retroviral vectors can be made target specific by inserting, for example, a polynucleotide encoding a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome to allow target specific. delivery of the retroviral vector containing an oligonucleotide IMC.

### Pharmaceutical Compositions of Multimers and Multimer/IMC

The invention encompasses all pharmaceutical compositions comprising M2e multimers, M2e/IMC multimers, M2e/NP multimers, and M2e/NP/IMC multimers. Pharmaceutically acceptable carriers preferred for use with the IMC of the invention may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. A composition of multimer or multimer/IMC may also be lyophilized using means well known in the art, for subsequent reconstitution and use according to the invention. Alternatively. if the multimer or multimer/IMC are being used in combination with vaccines that are in liquid form (e.g., TIV), then the multimer or multimer/IMC could be formulated as a liquid as well.

Absorption promoters, detergents and chemical irritants (e.g., keritinolytic agents) can enhance transmission of an IMC composition into a target tissue. For reference concerning general principles regarding absorption promoters and detergents which have been used with success in mucosal delivery of organic and peptide-based drugs, see Chien, Novel Drug Delivery Systems, Ch. 4 (Marcel Dekker, 1992).

Examples of suitable nasal absorption promoters in particular are set forth at Chien, supra at Ch. 5, Tables 2 and 3; milder agents are preferred. Suitable agents for use in the method of this invention for mucosal/nasal delivery are also described in Chang, et al., Nasal Drug Delivery, “Treatise on Controlled Drug Delivery”, Ch. 9 and Table 3-4B thereof, (Marcel Dekker, 1992). Suitable agents which are known to enhance absorption of drugs through skin are described in Sloan, Use of Solubility Parameters from-Regular Solution Theory to Describe Partitioning-Driven Processes, Ch. 5, “Prodrugs: Topical and Ocular Drug Delivery” (Marcel Dekker, 1992), and at places elsewhere in the text.

Pharmaceutical compositions can also include vaccines which are formulated for use to induce an immune response to influenza virus. In one aspect, the invention provides a vaccine comprising a composition of a multimer comprising at least two copies of M2e. The vaccine may also additionally include NP. In one embodiment, the vaccine contains a composition that comprises a fusion protein comprising NP and at least 2 copies of M2e. These vaccines may also optionally include an IMC in a manner described herein. Examples of IMC which may be used include, but are not limited to 1018 ISS, 7909 and other type B oligos, CICs such as C295 and others, type C oligos such as C792 and others.

The vaccines can also include a carrier as described here. Examples of carriers which may be used include, but are not limited to, alum, microparticles, liposomes, and nanoparticles. The vaccines of the invention further can also contain one or more components of monovalent, divalent or one trivalent inactivated influenza vaccine (TIV). An example of monovalent vaccine which may be used is a H5 pandemic vaccine. Non-limiting examples of TIV which may be used are Fluzone, Fluvirin, Fluarix, FluLaval, FluBlok, FluAd, Influvac, and Fluvax.

### Methods and Routes for Administration of Multimer or Multimer/IMC to an Individual

The multimer or multimer/IMC compositions and vaccines of the invention are administered to an individual using any available method and route suitable for drug delivery. In a preferred embodiment, the multimer or multimer/IMC compositions and vaccines of the invention are administered by injection with a needle, as with other standard influenza vaccines. In one embodiment, the multimers, with or without IMC, is delivered to the upper and/or lower respiratory tract by any delivery means known to one of skill in the art. In a preferred embodiment, multimers with or without IMC are delivered as a vaccine. Optionally the multimers are administered with other monovalent, divalent or trivalent influenza vaccines. Another possible method of delivery is intranasal delivery. Another possible method of multimer or multimer/IMC delivery is by insufflation. Other methods of administration include ex vivo methods (e.g., delivery of cells incubated or transfected with multimer or multimer/IMC) as well as systemic or localized routes. One of ordinary skill in the art will appreciate that methods and routes of delivery which direct the IMC into the individual should avoid degradation of the IMC in vivo.

Intranasal administration means are particularly useful in addressing respiratory disorders such as influenza virus infection. Such means include inhalation of aerosol suspensions of the multimer or multimer/IMC compositions of the invention. Nebulizer devices suitable for delivery of multimer or multimer/IMC compositions to the nasal mucosa, trachea and bronchioli are well-known in the art and will therefore not be described in detail here. For general review in regard to intranasal drug delivery, those of ordinary skill in the art may wish to consult Chien, Novel Drug Delivery Systems, Ch. 5 (Marcel Dekker, 1992).

In one aspect, the multimer or multimer/IMC compositions and vaccines of the invention are administered to an individual in need thereof at dose of about 0.1 μg to about 5 mg, more preferably between 0.25 μg and 3 mg, even more preferably between 0.5 μg and 1 mg, even more preferably between 0.75 μg and 500 μg, even more preferably between 1 μg and 100 μg.

### Kits for Use in Practicing the Methods of the Invention

For use in the methods described above, kits are also provided by the invention. Such kits may include any or all of the following: multimers of M2e, M2e/NP, M2e/IMC (conjugated or unconjugated); M2e//NP/IMC (conjugated or unconjugated) a pharmaceutically acceptable carrier (may be pre-mixed with the IMC) or suspension base for reconstituting lyophilized multimers or multimer/IMC; additional medicaments; a sterile vial for each IMC and additional medicament, or a single vial for mixtures thereof, devices) for use in delivering multimers or multimer/IMC to a individual; assay reagents for detecting indicia that the immunomodulatory effects sought have been achieved in treated individuals, instructions for how to and when administer the multimers or multimer/IMC and a suitable assay device.

In addition, the invention also provides for kits comprising M2e multimers or M2e/NP multimers (with or without conjugation to an IMC) and one or more components of an influenza vaccine (e.g., TIV).

### Methods of the Invention

The compositions and/or vaccines of the invention can be used to induce an immune response to combat infection with different strains of influenza virus. Exemplary strains of influenza virus which may be targets of the immune response are shown in FIG. 1. The consensus sequence of human, avian and swine M2e and their variants are shown in FIG. 1. The consensus sequence of NP and its variants are shown in FIG. 2. The immune response against influenza virus may be humoral response or cellular immune response or a combination of both responses. An immune response in animals or cell populations can be detected in any number of ways, including a increased expression of one or more of IFN-γ, IFN-α, IL-2, IL-12, TNF-α, IL-6, IL-4, IL-5, IP-10, ISG-54K, MCP-1, or a change in gene expression profile characteristic of immune stimulation as well as responses such as B cell proliferation and dendritic cell maturation, The ability to stimulate an immune response in a cell population has a number of uses, e.g., in an assay system for immunosuppressive agents.

Analysis (both qualitative and quantitative) of the immune response to multimers can be by any method known in the art, including, but not limited to, measuring antigen-specific antibody production (including measuring specific antibody+subclasses), activation of specific populations of lymphocytes such as CD4+ T cells, NK cells or CTLs, production of cytokines such as IFN-γ, IFN-α, IL-2, IL-4, IL-5, IL-10 or IL-12 and/or release of histamine. Methods for measuring specific antibody responses include enzyme-linked immunosorbent assay (ELISA) and are well known in the art. Measurement of numbers of specific types of lymphocytes such as CD4+ T cells can be achieved, for example, with fluorescence-activated cell sorting (FACS). Cytotoxicity and CTL assays can be performed for instance as described in Raz et al. (1994) Proc. Natl. Acad. Sci. USA 91:95 19-9523 and Cho et al. (2000). Cytokine concentrations can be measured, for example, by ELISA. These and other assays to evaluate the immune response to an immunogen are well known in the art. See, for example, SELECTED METHODS IN CELLULAR IMMUNOLOGY (1980) Mishell and Shiigi, eds., W.H. Freeman and Co.

Preferably, a Th1-type response is stimulated, i.e., elicited and/or enhanced. With reference to the invention, stimulating a Th1-type immune response can be determined in vitro or ex vivo by measuring cytokine production from cells treated with multimers or multimers/IMC as compared to control cells not treated with multimers or multimers/IMC. Methods to determine the cytokine production of cells include those methods described herein and any known in the art. The type of cytokines produced in response to multimers or multimers/IMC treatment indicate a Th1-type or a Th2-type biased immune response by the cells. As used herein, the term “Th1-type biased” cytokine production refers to the measurable increased production of cytokines associated with a Th1-type immune response in the presence of a stimulator as compared to production of such cytokines in the absence of stimulation. Examples of such Th1-type biased cytokines include, but are not limited to, IL-2, IL-12, IFN-γ, IFN-α, and TNF-α. In contrast, “Th2-type biased cytokines” refers to those associated with a Th2-type immune response, and include, but are not limited to, IL-4, IL-5, and IL-13. Cells useful for the determination of multimers or multimers/IMC activity include cells of the immune system, primary cells isolated from a individual and/or cell lines, preferably APCs and lymphocytes (e.g., macrophages and T cells) and splenocytes.

Stimulating a Th1-type immune response can also be measured in an individual treated with a multimers or multimers/IMC can be determined by any method known in the art including, but not limited to: (1) INF-γ measured before and after treatment with multimers or multimers/IMC; (2) an increase in levels of IL-12, IL-18 and/or IFN ( [symbol] , [symbol] or γ) before and after treatment with multimers or multimers/IMC; (3) “Th1-type biased” antibody production in a multimers or multimers/IMC treated individual as compared to a control treated without multimers or multimers/IMC. A variety of these determinations can be made by measuring cytokines made by splenocytes, APCs and/or lymphocytes, in vitro or ex vivo using methods described herein or any known in the art. Some of these determinations can be made by measuring the class and/or subclass of influenza-specific antibodies using methods described herein or any known in the art.

The class and/or subclass of antigen-specific (i.e., influenza-specific) antibodies produced in response to multimers or multimers/IMC treatment indicate a Th1-type or a Th2-type biased immune response by the cells. As used herein, the term “Th1-type biased” antibody production refers to the measurable increased production of antibodies associated with a Th1-type immune response (i.e., Th1-associated antibodies). One or more Th1 associated antibodies may be measured. Examples of such Th1-type biased antibodies include, but are not limited to, human IgG1 and/or IgG3 (see, e.g., Widhe et al. (1998) Scand. J. Immunol. 1. Immunol. 47:575-581 and de Martino et al. (1999) Ann. Allergy Asthma Immunol. 83:160-164) and murine IgG2a. In contrast, “Th2-type biased antibodies” refers to those associated with a Th2-type immune response, and include, but are not limited to, human IgG2, IgG4 and/or IgE (see, e.g., Widhe et al. (1998) and de Martino et al. (1999)) and murine IgG1 and/or IgE.

The Th1-type biased cytokine induction which occurs as a result of administration of multimers or multimers/IMC produces enhanced cellular immune responses, such as those performed by NK cells, cytotoxic killer cells, Th1 helper and memory cells. These responses are particularly beneficial for use in protective or therapeutic vaccination against various strains of influenza viruses. As such, the compositions and vaccines of the invention may be used an a universal vaccine to vaccinate against multiple strains of influenza viruses.

The compositions and vaccines of multimers and/or multimer/IMC can also be used for ameliorating one or more symptoms associated with infection with influenza virus in an individual. This is accomplished by administering to the individual a vaccine comprising a multimer of an extracellular domain of influenza matrix protein (M2e) wherein the multimer is capable of inducing an immune response in an individual. Symptoms associated with infection with influenza virus include, but are not limited to, body aches (especially joints and throat), coughing and sneezing, extreme coldness and fever, fatigue, headache, irritated watering eyes, nasal congestion, nausea and vomiting, and reddened eyes, skin (especially face), mouth, throat and nose. In one embodiment, the vaccine further comprises NP. In other embodiments of the invention, the vaccine further comprises an IMC.

In another aspect of the invention, the compositions and vaccines of the invention provide for methods for reducing the likelihood of infection with influenza virus in an individual by administering to the individual: (a) a vaccine comprising at least two copies of M2e and (b) one or more components of monovalent, divalent or trivalent inactivated vaccines (TIV). Examples of TIV include, but are not limited to, Fluzone, Fluvirin, Fluarix, FluLaval, FluBlok, FluAd, Influvac, and Fluvax. In some embodiments, the vaccine further comprises NP as described above. In other embodiments, the vaccine further comprises an IMC in any of the manners described herein and known in the art.

The following examples are provided to illustrate aspects of the invention but are not intended to limit the invention in any manner.

### EXAMPLES

### Example 1

### Construction of 8×(M2e)-NP-6×HisTag (N8-his tagged)

A construct containing 8 copies of the extracellular portion of the matrix 2 (M2e) gene fused 5′ to the nucleoprotein gene was made and expressed in E. coli. The nucleotide sequence of this construct is as follows (The underlined sequences indicate the restriction enzyme sites used to clone the gene construct into the plasmid vector.):

(SEQ ID NO: 1)

CATATGTCTCTGTTAACGGAAGTCGAGACACCCATCCGGAATGAGTGGGG
TTCCCGTAGTAATGATAGTTCGGATAGCTTACTGACCGAGGTTGAAACAC
CTATTCGTAACGAATGGGGTAGCCGGTCAAATGACTCGAGCGATTCGTTG
TTGACCGAAGTAGAGACCCCAATCCGCAATGAATGGGGCTCCCGGAGTAA
CGATAGCAGCGACTCCTTACTGACGGAGGTGGAAACGCCCATCCGTAACG
AGTGGGGTTCTAGAAGTAACGATTCCTCGGATAGCTTATTAACAGAAGTC
GAAACGCCTATTCGCAATGAATGGGGTTCGCGTTCGAATGATTCCAGTGA
TAGCCTGTTAACGGAAGTTGAAACTCCGATCCGTAATGAGTGGGGCAGCC
GTAGCAACGACTCGAGCGACTCCCTGCTCACTGAGGTTGAGACACCAATC
CGGAACGAATGGGGCTCGCGCTCGAACGATTCTTCCGATTCTCTGCTGAC
CGAAGTAGAAACTCCTATTCGTAATGAATGGGGTTCCCGTTCCAATGATA
GCAGCGATATGGCTTCCCAGGGTACTAAACGTAGCTATGAACAGATGGAA
ACCGATGGTGAACGTCAGAACGCGACTGAAATCCGTGCTAGCGTAGGTAA
AATGATCGGTGGTATCGGTCGTTTCTACATCCAGATGTGCACTGAACTTA
AACTTAGCGACTATGAAGGTCGTCTGATCCAGAATTCTCTGACCATTGAA
CGTATGGTTCTTAGCGCGTTTGATGAACGTCGTAACAAATACCTTGAAGA
ACACCCGTCTGCTGGTAAAGACCCTAAAAAAACTGGTGGTCCGATCTATC
GTCGTGTTAACGGTAAATGGATGCGTGAACTGATCCTGTATGACAAAGAA
GAAATCCGTCGTATTTGGAGACAGGCTAACAATGGTGATGACGCGACGGC
TGGACTGACCCACATGATGATTTGGCACAGCAACCTGAACGATGCGACCT
ACCAGCGTACCCGTGCGTTAGTACGTACCGGTATGGACCCGCGTATGTGT
AGCCTGATGCAAGGTAGCACTCTGCCTCGTCGTTCTGGTGCGGCTGGTGC
GGCGGTTAAAGGTGTGGGTACTATGGTTATGGAACTGGTTCGTATGATTA
AACGTGGTATCAACGATCGTAACTTTTGGCGTGGTGAAAATGGTCGTAAA
ACCCGTATCGCGTATGAACGTATGTGCAACATCCTTAAAGGTAAATTTCA
GACCGCAGCGCAGAAAGCTATGATGGACCAGGTTCGTGAATCTCGTAATC
CGGGTAATGCTGAGTTCGAAGACCTGACCTTCCTGGCTCGTTCTGCACTG
ATCCTGCGTGGTAGCGTAGCGCACAAATCTTGCCTGCCAGCGTGTGTTTA
CGGTCCGGCGGTTGCTAGCGGTTATGACTTCGAACGTGAAGGTTACTCTT
TGGTTGGTATTGACCCGTTCCGACTGCTCCAGAACTCCCAGGTTTACTCT
CTGATCCGTCCTAACGAAAACCCGGCGCATAAATCTCAGTTAGTTTGGAT
GGCTTGTCACTCTGCGGCGTTTGAAGAGCTGCGTGTTCTGAGCTTCATTA
AAGGTACTAAAGTTCTGCCGCGTGGTAAACTGTCTACCCGTGGTGTTCAG
ATCGCTAGCAATGAAAACATGGAAACTATGGAATCTAGCACCCTAGAACT
GCGTAGTCGTTATTGGGCGATCCGTACCCGTAGCGGTGGTAATACCAACC
AGCAGCGTGCGAGCGCGGGTCAGATTAGCATCCAGCCGACCTTTAGCGTT
CAGCGTAACCTGCCGTTTGACCGTACCACCATCATGGCTGCGTTTAACGG
TAACACTGAAGGTCGTACCAGTGACATGCGTACTGAAATCATCCGTATGA
TGGAATCTGCTCGACCGGAAGACGTGAGCTTTCAGGGTCGTGGTGTTTTT
GAACTTAGCGATGAAAAAGCTGCTAGCCCGATCGTTCCTAGCTTTGACAT
GTCTAACGAAGGTAGCTACTTCTTCGGTGACAACGCTGAGGAATATGACA
ACCATCATCACCATCACCATTAATAAGGATCC

The following is the protein sequence of the fusion protein:

| | |
|---|---|
| MSLLTEVETPIRNEWGSRSNDSSDSLLTEVETPIRNEWGSRSNDSSDSLLTEVETPIRNEWGS | (SEQ ID NO: 2) |
| RSNDSSDSLLTEVETPIRNEWGSRSNDSSDSLLTEVETPIRNEWGSRSNDSSDSLLTEVETPIR | |
| NEWGSRSNDSSDSLLTEVETPIRNEWGSRSNDSSDSLLTEVETPIRNEWGSRSNDSSDMASQ | |
| GTKRSYEQMETDOERQNATEIRASVGKMIGGIGRFYIQMCTELKLSDYEGRLIQNSLTIERM | |
| VLSAFDERRNKYLEEHPSAGKDPKKTGGPIYRRVNGKWMRELILYDKEEIRRIWRQANNG | |
| DDATAGLTHMMIWHSNLNDATYQRTRALVRTGMDPRMCSLMQGSTLPRRSGAAGAAVK | |
| GVGTMVMELVRMIKRGINDRNFWRGENGRKTRIAYERMCNILKGKFQTAAQKAMMDQV | |
| RESRNPGNAEFEDLTFLARSALILRGSVAHKSCLPACVYGPAVASGYDFEREGYSLVGIDPF | |
| RLLQNSQVYSLIRPNENPAHKSQLVWMACHSAAFEDLRVLSFIKGTKVLPRGKLSTRGVQI | |
| ASNENMETMESSTLELRSRYWAIRTRSGGNTNQQRASAGQISIQPTFSVQRNLPFDRTTIMA | |
| AFNGNTEGRTSDMRTEIIRMMESARPEDVSFQGRGVFELSDEKAASPIVPSFDMSNEGSYFF | |
| GDNAEEYDNHHHHHH | |

### Example 2

### Construction of 4×(M2e)-NP4×(M2e)-6×HisTag (N4/C4-his tagged)

A construct containing 4 copies of the M2e gene fused both 5′ and 3′ to the nucleoprotein gene was made and expressed in E. coli. The nucleotide sequence of this construct is as follows:

(SEQ ID NO: 3)

CATATGAGCCTGTTAACCGAAGTCGAGACGCCTATTCGTAATGAATGGGG
CAGTCGGTCGAACGATAGCTCGGATAGCCTGCTGACGGAGGTGGAAACCC
CGATCCGTAACGAGTGGGGCTCTCGTAGTAACGACTCGAGCGATAGCTTA
CTGACTGAAGTTGAAACTCCAATTCGCAATGAGTGGGGTAGCCGCAGCAA
TGATAGCAGTGATAGCTTATTAACGGAAGTTGAAACGCCTATCCGGAACG
AATGGGGTTCTAGAAGCAACGATAGTAGCGATATGGCTTCCCAGGGTACT
AAACGTAGCTATGAACAGATGGAAACCGATGGTGAACGTCAGAACGCGAC
TGAAATCCGTGCTAGCGTAGGTAAAATGATCGGTGGTATCGGTCGTTTCT
ACATCCAGATGTGCACTGAACTTAAACTTAGCGACTATGAAGGTCGTCTG
ATCCAGAATTCTCTGACCATTGAACGTATGGTTCTTAGCGCGTTTGATGA
ACGTCGTAACAAATACCTTGAAGAACACCCGTCTGCTGGTAAAGACGCTA
AAAAAACTGGTGGTCCGATCTATCGTCGTGTTAACGGTAAATGGATGCGT
GAACTGATGCTGTATGACAAAGAAGAAATCCGTCGTATTTGGAGACAGGC
TAACAATGGTGATGACGGGACCGCTGGACTGACCCACATGATGATTTGGC
ACAGCAACCTGAACGATGCGACCTACCAGCGTACCCGTGCGTTAGTACGT
ACCGGTATGGACCCGCGTATGTGTAGCCTGATGCAAGGTAGCACTCTGCC
TCGTCGTTCTGGTGCGGCTGGTGCGGCGGTTAAAGGTGTGGGTACTATGG
TTATGGAACTGGTTCGTATGATTAAACGTGGTATCAACGATCGTAACTTT
TGGCGTGGTGAAAATGGTCGTAAAACCCGTATCGCGTATGAACGTATGTG
CAACATCCTTAAAGGTAAATGTCAGACCGCAGCGCAGAAAGCTATGATGG
ACCAGGTTCGTGAATCTCGTAATCCGGGTAATGCTGAGTTCGAAGACCTG
ACCTTCCTGGCTCGTTCTGCACTGATCCTGCGTGGTAGCGTAGCGCACAA
ATCTTGCCTGCCAGCGTGTGTTTACGGTCCGGCGGTTGCTAGCGGTTATG
ACTTCGAACGTGAAGGTTACTCTTTGGTTGGTATTGACCCGTTCCGACTG
CTCCAGAACTCCCAGGTTTACTCTCTGATCCGTCCTAACGAAAACCCGGC
GCATAAATCTCAGTTAGTTTGGATGGCTTGTCACTCTGCGGCGTTTGAAG
ACCTGCGTGTTCTGAGCTTCATTAAAGGTACTAAAGTTCTGCCGCGTGGT
AAACTGTCTACCCGTGGTGTTCAGATCGCTAGCAATGAAAACATGGAAAC
TATGGAATCTAGCACCCTAGAACTGCGTAGTCGTTATTGGGCGATCCGTA
CCCGTAGCGGTGGTAATACCAACCAGCAGCGTGCGAGCGCGGGTCAGATT
AGCATCCAGCCGACCTTTAGCGTTCAGCGTAACCTGCCGTTTGACCGTAC
CACCATCATGGCTGCGTTTAACGGTAACACTGAAGGTCGTACCAGTGACA
TGCGTACTGAAATCATCCGTATGATGGAATCTGCTCGACCGGAAGACGTG
AGCTTTCAGGGTCGTGGTGTTTTTGAACTTAGCGATGAAAAAGCTGCTAG
CCCGATCGTTCCTAGCTTTGACATGTCTAACGAAGGTAGCTACTTCTTCG
GTGACAACGCTGAGGAATATGACAACTCTCTGTTGACTGAAGTAGAGACT
CCAATTCGTAACGAATGGGGTAGCCGTTCTAACGACTCTTCCGACTCTCT
GCTCACCGAGGTTGAAACCCCGATTCGCAATGAATGGGGCTCGCGTTCCA
ATGACTCGAGCGATTCTCTCCTGACGGAGGTTGAGACGCCTATCCGTAAT
GAGTGGGGTTCCCGGAGCAATGATTCTTCTGATTCTCTGCTGACTGAAGT
CGAAACCCCGATTCGGAACGAGTGGGGCAGTCGTTCAAATGACTCGTCGG
ACCATCATCATCACCATCATTAATAAGGATCC

The following is the protein sequence of the fusion protein:

(SEQ ID NO: 4)

MSLLTEVETPIRNEWGSRSNDSSDSLLTEVETPIRNEWGSRSNDSSDSLL
TEVETPIRNEWGSRSNDSSDSLLTEVETPIRNEWGSRSNDSSDMASQGTK
RSYEQMETDGERQNATEIRASVGKMIGGIGRFYIQMCTELKLSDYEGRLI
QNSLTIERMVLSAFDERRNKYLEEHPSAGKDPKKTGGPIYRRVNGKWMRE
LILYDKEEIRRIWRQANNGDDATAGLTHMMIWHSNLNDATYQRTRALVRT
GMDPRMCSLMQGSTLPRRSGAAGAAVKGVGTMVMELVRMIKRGINDRNFW
RGENGRKTRIAYERMCNILKGKFQTAAQKAMMDQVRESRNPGNAEFEDLT
FLARSALILRGSVAHKSCLPACVYGPAVASGYDFEREGYSLVGIDPFRLL
QNSQVYSLIRPNENPAHKSQLVWMACHSAAFEDLRVLSFIKGTKVLPRGK
LSTRGVQIASNENMETMESSTLELRSRYWAIRTRSGGNTFNQQRASAGQI
SIQPTFSVQRNLPFDRTTIMAAFNGNTEGRTSDMRTEIIRMMESARPEDV
SEQGRGVFELSDEKAASPIVPSFDMSNEGSYFFGDNAEEYDNSLLTEVET
PIRNEWGSRSNDSSDSLLTEVETPIRNEWGSRSNDSSDSLLTEVETPIRN
EWGSRSNDSSDSLLTEVETPIRNEWGSRSNDSSDHHHHHH

### Example 3

### Construction of 4×(M2e)-NP-6×HisTag (N4-his tagged)

A construct containing 4 copies of the M2e gene fused 5′ to the nucleoprotein gene was made and expressed in E. coli. The nucleotide sequence of this construct is as follows:

(SEQ ID NO: 5)

CATATGAGCCTGTTAACGGAGGTGGAAACTCCAATTCGGAATGAATGGGG
TTCGCGCAGCAATGATAGCTGGGATAGCTTACTGACCGAAGTCGAAACAC
CCATCCGTAACGAATGGGGCAGCCGTAGCAACGACTCGAGCGACTCCCTG
CTCACTGAGGTTGAGACCCCGATCCGCAATGAGTGGGGCTCGCGCTCGAA
CGATTCTTCCGA1TCTCTGCTGACCGAAGTAGAAACTCCTATTCGTAATG
AATGGGGTTCCCGTTCCAATGATAGCAGCGATATGGCTTCCCAGGGTACT
AAACGTAGCTATGAACAGATGGAAACCGATGGTGAACGTCAGAACGCGAC
TGAAATCCGTGCTAGCGTAGGTAAAATGATCGGTGGTATCGGTCGTTTCT
ACATCCAGATGTGCACTGAACTTAAACTTAGCGACTATGAAGGTCGTCTG
ATCCAGAATTCTCTGACCATTGAACGTATGGTTCTTAGCGCGTTTGATGA
ACGTCGTAACAAATACCTTGAAGAACACCCGTCTGCTGGTAAAGACCCTA
AAAAAACTGGTGGTCCGATCTATCGTCGTGTTAACGGTAAATGGATGCGT
GAACTGATCCTGTATGACAAAGAAGAAATCCGTCGTATTTGGAGACAGGC
TAACAATGGTGATGACGCGACCGCTGGACTGACCCACATGATGATTTGGC
ACAGCAACCTGAACGATGCGACCTACCAGCGTACCCGTGCGTTAGTACGT
ACCGGTATGGACGCGCGTATGTGTAGCCTGATGCAAGGTAGCACTCTGCC
TCGTCGTTCTGGTGCGGCTGGTGCGGCGGTTAAAGGTGTGGGTAGTATGG
TTATGGAACTGGTTCGTATGATTAAACGTGGTATCAACGATCGTAACTTT
TGGCGTGGTGAAAATGGTCGTAAAACCCGTATCGCGTATGAACGTATGTG
CAACATCCTTAAAGGTAAATTTCAGACCGCAGCGCAGAAAGCTATGATGG
ACCAGGTTCGTGAATCTCGTAATCCGGGTAATGCTGAGTTCGAAGACCTG
ACCTTCCTGGCTCGTTCTGCACTGATCCTGCGTGGTAGCGTAGCGCACAA
ATCTTGCCTGCCAGCGTGTGTTTACGGTCCGGCGGTTGCTAGCGGTTATG
ACTTCGAACGTGAAGGTTACTCTTTGGTTGGTATTGACCCGTTCCGACTG
CTCCAGAACTCCCAGGTTTACTCTCTGATCCGTCCTAACGAAAACCCGGC
GCATAAATCTCAGTTAGTTTGGATGGCTTGTCACTCTGCGGCGTTTGAAG
ACCTGCGTGTTCTGAGCTTCATTAAAGGTACTAAAGTTCTGCCGCGTGGT
AAACTGTCTACCCGTGGTGTTCAGATCGCTAGCAATGAAAACATGGAAAC
TATGGAATCTAGCACCCTAGAACTGCGTAGTCGTTATTGGGCGATCCGTA
CCCGTAGCGGTGGTAATACCAACCAGCAGCGTGCGAGCGCGGGTCAGATT
AGCATCCAGCCGACCTTTAGCGTTCAGCGTAACCTGCCGTTTGACCGTAC
CACGATCATGGCTGCGTTTAACGGTAACACTGAAGGTCGTACCAGTGACA
TGCGTACTGAAATCATCCGTATGATGGAATCTGCTCGACCGGAAGACGTG
AGCTTTCAGGGTCGTGGTGTTTTTGAACTTAGCGATGAAAAAGCTGCTAG
CCCGATCGTTCCTAGCTTTGACATGTCTAACGAAGGTAGCTACTTCTTCG
GTGACAACGCTGAGGAATATGACAACCATCATCACCATCACCATTAATAA
GGATCC

The following is the protein sequence of the fusion protein:

(SEQ ID NO: 6)

MSLLTEVETPIRNEWGSRSNDSSDSLLTEVETPIRNEWGSRSNDSSDSLL
TEVETPIRNEWGSRSNDSSDSLLTEVETPIRNEWGSRSNDSSDMASQGTK
RSYEQMETDGERQNATEIRASVGKMIGGIGRFYIQMCTELKLSDYEGRLI
QNSLTIERMVLSAFDERRNKYLEEHPSAGKDPKKTGGPIYRRVNGKWMRE
LILYDKEEIRRIWRQANNGDDATAGLTHMMIWHSNLNDATYQRTRALVRT
GMDPRMCSLMQGSTLPRRSGAAGAAVKGVGTMVMELVRMIKRGINDRNFW
RGENGRKTRIAYERMCNILKGKFQTAAQKAMMDQVRESRNPGNAEFEDLT
FLARSALILRGSVAHKSCLPACVYGPAVASGYDFEREGYSLVG1DPFRLL
QNSQVYSLIRPNENPAHKSQLVWMACHSAAFEDLRVLSFIKGTKVLPRGK
LSTRGVQIASNENMETMESSTLELRSRYWAIRTRSGGNTNQQRASAGQIS
IQPTFSVQRNLPFDRTTIMAAFNGNTEGRTSDMRTEIIRMMESARPEDVS
FQGRGVFELSDEKAASPIVPSFDMSNEGSYFFGDNAEEYDNHHHHHH

### Example 4

### Construction of 4×(M2e-spacer)-NP-6×HisTag (N4s-his tagged)

A construct containing 4 copies of the M2e gene and a spacer fused 5′ to the nucleoprotein gene was made and expressed in E. coli. The nucleotide sequence of this construct is as follows:

(SEQ ID NO: 7)

CATATGTCCCTGCTGACGGAAGTAGAAACCCCAATTCGCAATGAATGGGG
CAGCCGTAGCAATGACTCTTCTGACGGTTCTGCGAGCGGTAGCTTGCTTA
CTGAAGTTGAAACTCCTATCCGTAACGAATGGGGTTCCCGTTCTAACGAC
TCGAGCGACGGCAGCGCGTCCGGTTCTCTGCTGACTGAGGTGGAGACTCC
GATTCGTAATGAGTGGGGTAGCCGCAGCAACGATTCTTCCGATGGCTCTG
CTTCTGGTTCCTTGTTGACCGAAGTTGAAACCCCTATCCGCAACGAATGG
GGCTCTCGCTGTAATGATAGCTCTGATGGTTCGGCTTCCGGCATGGCTTC
CCAGGGTACTAAACGTAGCTATGAACAGATGGAAACCGATGGTGAACGTC
AGAACGCGACTGAAATCCGTGCTAGCGTAGGTAAAATGATCGGTGGTATC
GGTCGTTTCTACATCCAGATGTGCACTGAACTTAAACTTAGCGACTATGA
AGGTCGTCTGATCCAGAATTCTCTGACCATTGAACGTATGGTTCTTAGCG
CGTTTGATGAACGTCGTAAGAAATACCTTGAAGAACACCCGTCTGCTGGT
AAAGACCCTAAAAAAACTGGTGGTCCGATCTATCGTCGTGTTAACGGTAA
ATGGATGCGTGAACTGATCCTGTATGACAAAGAAGAAATCCGTCGTATTT
GGAGACAGGCTAACAATGGTGATGACGCGACCGCTGGACTGACCCACATG
ATGATTTGGCACAGCAACCTGAACGATGCGACCTACCAGCGTACCCGTGC
GTTAGTACGTACCGGTATGGACCCGCGTATGTGTAGCCTGATGCAAGGTA
GCACTCTGCCTCGTCGTTCTGGTGCGGCTGGTGCGGCGGTTAAAGGTGTG
GGTACTATGGTTATGGAACTGGTTCGTATGATTAAACGTGGTATCAACGA
TCGTAACTTTTGGCGTGGTGAAAATGGTCGTAAAACCCGTATCGCGTATG
AACGTATGTGCAACATCCTTAAAGGTAAATTTCAGACCGCAGCGCAGAAA
GCTATGATGGACCAGGTTCGTGAATCTCGTAATCCGGGTAATGCTGAGTT
CGAAGACCTGACCTTCCTGGCTCGTTCTGCACTGATCCTGCGTGGTAGCG
TAGCGCACAAATCTTGCCTGCCAGCGTGTGTTTACGGTCCGGCGGTTGCT
AGCGGTTATGACTTCGAACGTGAAGGTTACTCTTTGGTTGGTATTGACCC
GTTCCGACTGCTCCAGAACTCCCAGGTTTACTCTCTGATCCGTCCTAACG
AAAACCCGGCGCATAAATCTCAGTTAGTTTGGATGGCTTGTCACTCTGCG
GCGTTTGAAGACCTGCGTGTTGTGAGCTTCATTAAAGGTACTAAAGTTCT
GCCGGGTGGTAAACTGTCTACCCGTGGTGTTGAGATCGCTAGCAATGAAA
ACATGGAAACTATGGAATCTAGCACCCTAGAACTGCGTAGTCGTTATTGG
GCGATGCGTACCCGTAGCGGTGGTAATACCAACCAGCAGCGTGCGAGCGC
GGGTCAGATTAGCATCCAGCCGACCTTTAGCGTTCAGCGTAACCTGCCGT
TTGACCGTACCACCATCATGGCTGCGTTTAACGGTAACACTGAAGGTCGT
ACCAGTGACATGCGTACTGAAATCATCCGTATGATGGAATCTGCTCGACC
GGAAGACGTGAGCTTTCAGGGTCGTGGTGTTTTTGAACTTAGCGATGAAA
AAGCTGCTAGCCCGATCGTTCCTAGCTTTGACATGTCTAACGAAGGTAGC
TACTTCTTCGGTGACAACGCTGAGGAATATGACAACCATCACCATCATCA
CCACTAATAAGGATCC

The following is the protein sequence of the fusion protein:

(SEQ ID NO: 8)

MSLLTEVETPIRNEWGSRSNDSSDGSASGSLLTEVETPIRNEWGSRSNDS
SDGSASGSLLTEVETPIRNEWGSRSNDSSDGSASGSLLTEVETPIRNEWG
SRSNDSSDGSASGMASQGTKRSYEQMETDGERQNATEIRASVGKMIGGIG
RFYIQMCTELKLSDYEGRLIQNSLTIERMVLSAFDERRNKYLEEHPSAGK
DPKKTGGPIYRRVNGKWMRELILYDKEEIRRIWRQANNGDDATAGLTHMM
IWHSNLNDATYQRTRALVRTGMDPRMCSLMQGSTLPRRSGAAGAAVKGVG
TMVMELVRMIKRGINDRNFWRGENGRKTRIAYERMCNILKGKFQTAAQKA
MMDQVRESRNPGNAEFEDLTFLARSALILRGSVAHKSCLPACVYGPAVAS
GYDFEREGYSLVGIDPFRLLQNSQVYSLIRPNENPAHKSQLVWMACHSAA
FEDLRVLSFIKGTKVLPRGKLSTRGVQIASNENMETMESSTLELRSRYWA
IRTRSGGNTNQQRASAGQISIQPTFSVQRNLPFDRTTLMAAFNGNTEGRT
SDMRTEIIRMMESARPEDVSFQGRGVFELSDEKAASPIVPSFDMSNEGSY
FFGDNAEEYDNHHHHHH

### Example 5

### Construction of 8×(M2e)-NP (N8 -non-his tagged)

A construct containing 8 copies of the M2e gene fused 5′ to the nucleoprotein gene was made and expressed in E. coli. The nucleotide sequence of this construct is as follows:

(SEQ ID NO: 9)

CATATGTCTCTGTTAACGGAAGTCGAGACACCCATCCGGAATGAGTGGGG
TTCCCGTAGTAATGATAGTTCGGATAGGTTACTGACCGAGGTTGAAACAC
CTATTCGTAACGAATGGGGTAGCCGGTCAAATGACTCGAGCGATTCGTTG
TTGACCGAAGTAGAGACCCCAATCCGCAATGAATGGGGCTCCCGGAGTAA
CGATAGCAGCGACTCCTTACTGACGGAGGTGGAAACGCCCATCCGTAACG
AGTGGGGTTCTAGAAGTAACGATTCCTCGGATAGCTTATTAACAGAAGTC
GAAACGCCTATTCGCAATGAATGGGGTTCGCGTTCGAATGATTCCAGTGA
TAGCCTGTTAACGGAAGTTGAAACTCCGATCCGTAATGAGTGGGGCAGCC
GTAGCAACGACTCGAGCGACTCCCTGCTCACTGAGGTTGAGACACCAATC
CGGAACGAATGGGGCTCGCGCTCGAACGATTCTTGCGATTCTCTGCTGAC
CGAAGTAGAAACTCCTATTCGTAATGAATGGGGTTCCCGTTCCAATGATA
GCAGCGATATGGCTTCCCAGGGTACTAAACGTAGCTATGAACAGATGGAA
ACCGATGGTGAACGTCAGAACGCGACTGAAATCCGTGCTAGCGTAGGTAA
AATGATCGGTGGTATCGGTCGTTTCTACATCCAGATGTGCACTGAACTTA
AACTTAGCGACTATGAAGGTCGTCTGATCCAGAATTCTCTGACCATTGAA
CGTATGGTTCTTAGCGCGTTTGATGAACGTCGTAACAAATACCTTGAAGA
ACACCCGTCTGCTGGTAAAGACCCTAAAAAAACTGGTGGTCCGATCTATC
GTCGTGTTAACGGTAAATGGATGCGTGAACTGATCCTGTATGACAAAGAA
GAAATCCGTCGTATTTGGAGACAGGCTAACAATGGTGATGACGCGACCGC
TGGACTGACCCACATGATGATTTGGCACAGCAACCTGAACGATGCGACCT
ACCAGCGTACCCGTGCGTTAGTACGTACCGGTATGGACCCGCGTATGTGT
AGCCTGATGGAAGGTAGCACTCTGCCTCGTCGTTCTGGTGCGGCTGGTGC
GGCGGTTAAAGGTGTGGGTACTATGGTTATGGAACTGGTCGTATGATTAA
ACGTGGTATCAACGATCGTAACTTTTGGCGTGGTGAAAATGGTCGTAAAA
CGCGTATCGCGTATGAACGTATGTGCAACATCCTTAAAGGTAAATTTCAG
ACCGCAGCGCAGAAAGCTATGATGGACCAGGTTCGTGAATCTCGTAATCC
GGGTAATGCTGAGTTCGAAGAGGTGACGTTCCTGGGTCGTTCTGCACTGA
TCCTGCGTGGTAGCGTAGCGCACAAATCTTGCCTGCCAGCGTGTGTTTAC
GGTCCGGCGGTTGCTAGCGGTTATGACTTCGAACGTGAAGGTTACTCTTT
GGTTGGTATTGACCCGTTCCGACTGCTCCAGAACTCCCAGGTTTACTCTC
TGATCCGTCCTAACGAAAACCCGGCGCATAAATCTCAGTTAGTTTGGATG
GCTTGTCACTCTGCGGCGTTTGAAGACCTGCGTGTTCTGAGCTTCATTAA
AGGTACTAAAGTTCTGCCGCGTGGTAAACTGTCTACCCGTGGTGTTCAGA
TCGCTAGCAATGAAAACATGGAAACTATGGAATCTAGCACCCTAGAACTG
CGTAGTCGTTATTGGGCGATCCGTACCCGTAGCGGTGGTAATACCAACCA
GCAGCGTGGGAGCGCGGGTCAGATTAGCATCCAGCCGACCTTTAGCGTTC
AGCGTAACCTGCCGTTTGACCGTACCACCATCATGGCTGCGTTTAACGGT
AACACTGAAGGTCGTACCAGTGACATGCGTACTGAAATCATCCGTATGAT
GGAATCTGCTCGACCGGAAGACGTGAGCTTTCAGGGTCGTGGTGTTTTTG
AACTTAGCGATGAAAAAGCTGCTAGCCCGATCGTTCCTAGCTTTGACATG
TCTAACGAAGGTAGCTACTTCTTCGGTGACAACGCTGAGGAATATGACAA
CTAATAAGGATCC

The following is the protein sequence of the fusion protein:

(SEQ ID NO: 10)

MSLLTEVETPIRNEWGSRSNDSSDSLLTEVETPIRNEWGSRSNDSSDSLL
TEVETPIRNEWGSRSNDSSDSLLTEVETPIRNEWGSRSNDSSDSLLTEVE
TPIRNEWGSRSNDSSDSLLTEVETPIRNEWGSRSNDSSDSLLTEVETPIR
NEWGSRSNDSSDSLLTEVETPIRNEWGSRSNDSSDMASQGTKRSYEQMET
DGERQNATEIRASVGKMIGGIGRFYIQMGTELKLSDYEGRLIQNSLTIER
MVLSAFDERRNKYLEEHPSAGKDPKKTGGPIYRRVNGKWMRELILYDKEE
IRRIWRQANNGDDATAGLTHMMIWHSNLNDATYQRTRALVRTGMDPRMCS
LMQGSTLPRRSGAAGAAVKGVGTMVMELVRMIKRGINDRNFWRGENGRKT
RLAYERMCNILKGKFQTAAQKAMMDQVRESRNPGNAEFEDLTFLARSALI
LRGSVAHKSCLPACVYGPAVASGYDFEREGYSLVGIDPFRLLQNSQVYSL
IRPNENPAHKSQLVWMACHSAAFEDLRVLSFIKGTKVLPRGKLSTRGVQI
ASNENMETMESSTLELRSRYWAIRTRSGGNTNQQRASAGQISIQPTFSVQ
RNLPFDRTTIMAAFNGNTEGRTSDMRTEIIRMMESARPEDVSFQGRGVFE
LSDEKAASPIVPSFDMSNEGSYFFGDNAEEYDN

### Example 6

### Construction of 4×(M2e)-NP-4×(M2e) (N4/C4 - non-his tagged)

A construct containing 4 copies of the M2e gene fused both 5′ and 3′ to the nucleoprotein gene was made and expressed in E. coli. The nucleotide sequence of this construct is as follows:

(SEQ ID NO: 11)

CATATGAGCCTGTTAACCGAAGTCGAGACGCCTATTCGTAATGAATGGGG
CAGTCGGTCGAACGATAGCTCGGATAGCCTGCTGACGGAGGTGGAAACCC
CGATCCGTAACGAGTGGGGCTCTCGTAGTAACGACTCGAGCGATAGCTTA
CTGACTGAAGTTGAAACTCCAATTCGCAATGAGTGGGGTAGCCGCAGCAA
TGATAGCAGTGATAGCTTATTAACGGAAGTTGAAACGCCTATCCGGAACG
AATGGGGTTCTAGAAGCAACGATAGTAGCGATATGGCTTCCCAGGGTACT
AAACGTAGCTATGAACAGATGGAAACCGATGGTGAACGTCAGAACGCGAC
TGAAATCCGTGCTAGCGTAGGTAAAATGATCGGTGGTATCGGTCGTTTCT
ACATCCAGATGTGCACTGAACTTAAACTTAGCGACTATGAAGGTCGTCTG
ATCCAGAATTCTCTGACCATTGAACGTATGGTTCTTAGCGCGTTTGATGA
ACGTCGTAACAAATACCTTGAAGAACACCCGTCTGCTGGTAAAGACCCTA
AAAAAACTGGTGGTCCGATCTATCGTCGTGTTAACGGTAAATGGATGCGT
GAACTGATCCTGTATGACAAAGAAGAAATCCGTCGTATTTGGAGACAGGC
TAACAATGGTGATGACGCGACCGCTGGACTGACCCACATGATGATTTGGC
ACAGCAACCTGAACGATGCGACCTACCAGCGTACGCGTGCGTTAGTACGT
ACCGGTATGGACCCGCGTATGTGTAGCCTGATGCAAGGTAGCACTCTGCC
TCGTCGTTCTGGTGGGGCTGGTGGGGCGGTTAAAGGTGTGGGTACTATGG
TTATGGAACTGGTTCGTATGATTAAACGTGGTATCAACGATCGTAACTTT
TGGCGTGGTGAAAATGGTCGTAAAACCCGTATCGCGTATGAACGTATGTG
CAACATCCTTAAAGGTAAATTTCAGACCGCAGCGCAGAAAGCTATGATGG
ACCAGGTTCGTGAATCTCGTAATCCGGGTAATGCTGAGTTCGAAGACCTG
ACCTTCGTGGCTCGTTCTGCACTGATCCTGCGTGGTAGCGTAGCGCACAA
ATCTTGCCTGCCAGCGTGTGTTTACGGTCCGCCGGTTGCTAGCGGTTATG
ACTTCGAACGTGAAGGTTAGTCTTTGGTTGGTATTGACCCGTTCCGACTG
CTCCAGAACTCCCAGTGTTACTCTCTGATCCGTCCTAACGAAAACCCGGC
GCATAAATGTCAGTTAGTTTGGATGGCTTGTCACTCTGCGGCGTTTGAAG
ACCTGCGTGTTCTGAGCTTCATTAAAGGTACTAAAGTTCTGCCGCGTGGT
AAACTGTCTACCCGTGGTGTTCAGATCGCTAGCAATGAAAACATGGAAAC
TATGGAATCTAGCACCCTAGAACTGCGTAGTCGTTATTGGGCGATCCGTA
CCCGTAGCGGTGGTAATACCAACCAGCAGCGTGCGAGCGCGGGTCAGATT
AGCATCCAGCCGACCTTTAGCGTTGAGCGTAACCTGCCGTTTGACCGTAC
CACCATCATGGCTGCGTTTAACGGTAACACTGAAGGTCGTACCAGTGACA
TGCGTACTGAAATCATCCGTATGATGGAATCTGCTCGACCGGAAGACGTG
AGCTTTCAGGGTCGTGGTGTTTTTGAACTTAGCGATGAAAAAGCTGCTAG
CCCGATCGTTCCTAGCTTTGACATGTCTAACGAAGGTAGCTACTTCTTCG
GTGACAACGCTGAGGAATATGACAACTCTCTGTTGACTGAAGTAGAGACT
CCAATTCGTAACGAATGGGGTAGCCGTTCTAACGACTCTTCCGACTCTCT
GCTCACCGAGGTTGAAACCCCGATTCGCAATGAATGGGGCTCGCGTTCCA
ATGACTCGAGCGA1TCTCTCCTGACGGAGGTTGAGACGCCTATCCGTAAT
GAGTGGGGTTCCCGGAGCAATGATTCTTCTGATTCTCTGCTGACTGAAGT
CGAAACCCCGATTCGGAACGAGTGGGGGAGTCGTTCAAATGACTCGTCGG
ACTAATAAGGATCC

The following is the protein sequence of the fusion protein:

(SEQ ID NO: 12)

MSLLTEVETPIRNEWGSRSNDSSDSLLTEVETPIRNEWGSRSNDSSDSLL
TEVETPIRNEWGSRSNDSSDSLLTEVETPIRNEWGSRSNDSSDMASQGTK
RSYEQMETDGERQNATEIRASVGKMIGGIGRFYIQMCTELKLSDYEGRLI
QNSLTIERMVLSAFDERRNKYLEEHPSAGKDPKKTGGPIYRRVNGKWMRE
LILYDKEEIRRIWRQANNGODATAGLTHMMIWHSNLNDATYQRTRALVRT
GMDPRMCSLMQGSTLPRRSGAAGAAVKGVGTMVMELVRMIKRGINDRNFW
RGENGRKTRIAYERMCNILKGKFQTAAQKAMMDQVRESRNPGNAEFEDLT
FLARSALILRGSVAHKSCLPACVYGPAVASGYDFEREGYSLVQIDPFRLL
QNSQVYSLIRPNENPAHKSQLVWMACHSAAFEDLRVLSFIKGTKVLPRGK
LSTRGVQIASNENMETMESSTLELRSRYWAIRTRSGGNTNQQRASAGQIS
IQPTFSVQRNLPFDRTTIMAAFNGNTEGRTSDMRTEIIRMMESARPEDVS
FQGRGVFELSDEKAASPIVPSFDMSNEGSYFFGDNAEEYDNSLLTEVETP
IRNEWGSRSNDSSDSLLTEVETPIRNEWGSRSNDSSDSLLTEVETPIRNE
WGSRSNDSSDSLLTEVETPIRNEWGSRSNDSSD

### Example 7

### Construction of 4×M2e-NP (N4 -non-his tagged)

A construct containing 4 copies of the M2e gene fused 5′ to the nucleoprotein gene is made and expressed in E. coli. The nucleotide sequence of this construct is as follows:

(SEQ ID NO: 13)

CATATGAGCCTGTTAACGGAGGTGGAAACTCCAATTCGGAATGAATGGGG
TTCGCGCAGCAATGATAGCTCGGATAGCTTACTGACCGAAGTCGAAACAC
CCATCCGTAACGAATGGGGCAGGCGTAGCAACGACTCGAGCGACTCCCTG
CTCACTGAGGTTGAGACCCCGATCCGCAATGAGTGGGGCTCGCGCTCGAA
CGATTCTTCCGATTCTCTGCTGACCGAAGTAGAAACTCCTATTCGTAATG
AATGGGGTTCCCGTTCCAATGATAGCAGCGATATGGCTTCCCAGGGTACT
AAACGTAGCTATGAACAGATGGAAACCGATGGTGAACGTCAGAACGCGAC
TGAAATCCGTGCTAGCGTAGGTAAAATGATCGGTGGTATCGGTCGTTTCT
ACATCCAGATGTGCACTGAACTTAAACTTAGCGACTATGAAGGTCGTCTG
ATCCAGAATTCTCTGACCATTGAACGTATGGTTCTTAGCGCGTTTGATGA
ACGTCGTAACAAATACCTTGAAGAACACCCGTCTGCTGGTAAAGACCCTA
AAAAAACTGGTGGTCCGATCTATCGTCGTGTTAACGGTAAATGGATGCGT
GAACTGATCCTGTATGACAAAGAAGAAATCCGTCGTATTTGGAGACAGGC
TAACAATGGTGATGACGCGACCGCTGGACTGACCCACATGATGATTTGGC
ACAGCAACCTGAACGATGCGACCTACCAGCGTACCGGTGCGTTAGTACGT
ACCGGTATGGACCCGCGTATGTGTAGCCTGATGCAAGGTAGCACTCTGCC
TCGTCGTTCTGGTGCGGCTGGTGCGGCGGTTAAAGGTGTGGGTACTATGG
TTATGGAACTGGTTCGTATGATTAAACGTGGTATCAACGATCGTAACTTT
TGGCGTGGTGAAAATGGTCGTAAAACCCGTATCGCGTATGAACGTATGTG
CAACATCCTTAAAGGTAAATTTCAGACCGCAGCGCAGAAAGCTATGATGG
ACCAGGTTCGTGAATCTCGTAATCCGGGTAATGCTGAGTTCGAAGACCTG
ACCTTCCTGGCTCGTTCTGCACTGATCCTGCGTGGTAGCGTAGCGCACAA
ATCTTGCCTGCCAGCGTGTGTTTACGGTCCGGCGGTTGCTAGCGGTTATG
ACTTCGAACGTGAAGGTTACTCTTTGGTTGGTATTGACCCGTTCCGACTG
CTCCAGAACTCCCAGGTTTACTCTCTGATCCGTCCTAACGAAAACCCGGC
GCATAAATCTCAGTTAGTTTGGATGGCTTGTCACTCTGCGGCGTTTGAAG
ACCTGCGTGTTCTGAGCTTCATTAAAGGTACTAAAGTTCTGCCGCGTGGT
AAACTGTCTACCCGTGGTGTTCAGATCGCTAGCAATGAAAACATGGAAAC
TATGGAATCTAGCACCCTAGAACTGCGTAGTCGTTATTGGGCGATCCGTA
CCCGTAGCGGTGGTAATACCAACGAGCAGCGTGCGAGCGCGGGTCAGATT
AGCATCCAGCCGACCTTTAGCGTTCAGCGTAACCTGCCGTTTGACCGTAC
CACCATCATGGCTGCGTTTAACGGTAACACTGAAGGTCGTACCAGTGACA
TGCGTACTGAAATCATCCGTATGATGGAATCTGCTCGACCGGAAGACGTG
AGCTTTCAGGGTCGTGGTGTTTTTGAACTTAGCGATGAAAAAGCTGCTAG
CCCGATCGTTCCTAGCTTTGACATGTCTAACGAAGGTAGCTACTTCTTCG
GTGACAACGCTGAGGAATATGACAACTAATAAGGATCC

The following is the protein sequence of the fusion protein:

(SEQ ID NO: 14)

MSLLTEVETPIRNEWGSRSNDSSDSLLTEVETPIRNEWGSRSNDSSDSLL
TEVETPIRNEWGSRSNDSSDSLLTEVETPIRNEWGSRSNDSSDMASQGTK
RSYEQMETDGERQNATEIRASVGKMIGGIORFYIQMCTELKLSDYEGRLI
QNSLTIERMVLSAFDERRNKYLEEHPSAGKDPKKTGGPIYRRVNGKWMRE
LILYDKEEIRRIWRQANNGDDATAGLTHMMIWHSNLNDATYQRTRALVRT
GMDPRMCSLMQGSTLPRRSOAAGAAVKGVGTMVMELVRMIKRGINDRNFW
RGENGRKTRIAYERMCNILKGKFQTAAQKAMMDQVRESRNPGNAEFEDLT
FLARSALILRGSVAHKSCLPAGVYGPAVASGYDFEREGYSLVGIDPFRLL
QNSQVYSLIRPNENPAHKSQLVWMACHSAAFEDLRVLSFIKGTKVLPRGK
LSTRGVQIASNENMETMESSTLELRSRYWAIRTRSGGNTNQQRASAGQIS
IQPTFSVQRNLPFDRTTIMAAFNGNTEGRTSDMRTEIIRMMESARPEDVS
FQGRGVFELSDEKAASPIVPSFDMSNEGSYFFGDNAEEYDN

### Example 8

### Construction of 4×(M2e-spacer)-NP(N4s-non-his tagged)

A construct containing 4 copies of the M2e gene with a spacer fused 5′ to the nucleoprotein gene and nucleoprotein is made and expressed in E. coli. The nucleotide sequence of this construct is as follows:

(SEQ ID NO: 15)

CATATGTCCCTGCTGACGGAAGTAGAAACCCCAATTCGCAATGAATGGGG
CAGCCGTAGCAATGACTCTTCTGACGGTTCTGCGAGCGGTAGCTTGCTTA
CTGAAGTTGAAACTCCTATCCGTAACGAATGGGGTTCCCGTTCTAACGAC
TCGAGCGACGGCAGCGCGTCCGGTTCTCTGCTGACTGAGGTCGAGACTCC
GATTCGTAATGAGTGGGGTAGCCGCAGCAACGATTCTTCCGATGGCTCTG
CTTCTGGTTCCTTGTTGACCGAAGTTGAAACCCCTATCCGCAACGAATGG
GGCTCTCGCTCTAATGATAGCTCTGATGGTTCGGCTTCCGGCATGGCTTC
CCAGGGTACTAAACGTAGCTATGAACAGATGGAAACCGATGGTGAACGTC
AGAACGCGACTGAAATCCGTGCTAGCGTAGGTAAAATGATCGGTGGTATC
GGTCGTTTCTACATCCAGATGTGCACTGAACTTAAACTTAGCGACTATGA
AGGTCGTCTGATCCAGAATTCTCTGACCATTGAACGTATGGTTCTTAGCG
CGTTTGATGAACGTCGTAACAAATACCTTGAAGAACACCCGTCTGCTGGT
AAAGACCCTAAAAAAACTGGTGGTCCGATCTATCGTCGTGTTAACGGTAA
ATGGATGCGTGAACTGATCCTGTATGACAAAGAAGAAATCCGTCGTATTT
GGAGACAGGCTAACAATGGTGATGACGCGACCGCTGGACTGACCCACATG
ATGATTTGGCACAGCAACCTGAACGATGCGACCTACCAGCGTACCCGTGC
GTTAGTACGTACCGGTATGGACCCGCGTATGTGTAGCCTGATGCAAGGTA
GCACTCTGCCTCGTCGTTCTGGTGCGGCTGGTGCGGCGGTTAAAGGTGTG
GGTACTATGGTTATGGAACTGGTTCGTATGATTAAACGTGGTATCAACGA
TCGTAACTTTTGGCGTGGTGAAAATGGTCGTAAAACCCGTATCGCGTATG
AACGTATGTGCAACATCCTTAAAGGTAAATTTCAGACCGCAGCGCAGAAA
GCTATGATGGACCAGGTTCGTGAATCTCGTAATCCGGGTAATGCTGAGTT
CGAAGACCTGACCTTCCTGGCTCGTTCTGCACTGATCCTGCGTGGTAGCG
TAGCGCACAAATCTTGCCTGCCAGCGTGTGTTTACGGTCCGGCGGTTGCT
AGCGGTTATGACTTCGAACGTGAAGGTTACTCTTTGGTTGGTATTGACCC
GTTCCGACTGCTCCAGAACTCCCAGGTTTACTCTCTGATCCGTCCTAACG
AAAACCCGGCGCATAAATCTCAGTTAGTTGGATGGCTTGTCACTCTGCGG
CGTTTGAAGACCTGCGTGTTGTGAGCTTCATTAAAGGTACTAAAGTTCTG
CCGCGTGGTAAACTGTCTACCCGTGGTGTTGAGATCGCTAGCAATGAAAA
CATGGAAACTATGGAATCTAGCACCCTAGAACTGCGTAGTCGTTATTGGG
CGATCCGTACCCGTAGCGGTGGTAATACCAACCAGCAGCGTGCGAGCGCG
GGTCAGATTAGCATCCAGCCGACCTTTAGCGTTCAGCGTAACCTGCCGTT
TGACCGTACCACCATCATGGCTGCGTTTAACGGTAACACTGAAGGTCGTA
CCAGTGACATGCGTACTGAAATCATCCGTATGATGGAATCTGCTCGAGCG
GAAGACGTGAGCTTTCAGGGTCGTGGTGTTTTTGAACTTAGCGATGAAAA
AGCTGCTAGCCCGATCGTTCCTAGCTTTGACATGTCTAACGAAGGTAGCT
ACTTCTTCGGTGACAACGCTGAGGAATATGACAACTAATAAGGATCC

The following is the protein sequence of the fusion protein:

(SEQ ID NO: 16)

MSLLTEVETPIRNEWGSRSNDSSDGSASGSLLTEVETPIRNEWGSRSNDS
SDGSASGSLLTEVETPIRNEWGSRSNDSSDGSASGSLLTEVETPIRNEWG
SRSNDSSDGSASGMASQGTKRSYEQMETDGERQNATEIRASVGKMIGGIG
RFYIQMCTELKLSDYEGRLIQNSLTIERMVLSAFDERRNKYLEEHPSAGK
DPKKTGGPIYRRVNGKWMRELILYDKEEIRRIWRQANNGDDATAGLTHMM
IWHSNLNDATYQRTRALVRTGMDPRMCSLMQGSTLPRRSGAAGAAVKGVG
TMVMELVRMIKRGINDRNFWRGENGRKTRIAYERMCNILKGKFQTAAQKA
MMDQVRESRNPGNAEFEDLTFLARSALILRGSVAHKSCLPACVYGPAVAS
GYDFEREGYSLVGIDPFRLLQNSQVYSLIRPNENPAHKSQLVWMACHSAA
FEDLRVLSFIKGTKVLPRGKLSTRGVQIASNENMETMESSTLELRSRYWA
IRTRSGGNTNQQRASAGQISIQPTFSVQRNLPFDRTTIMAAFNGNTEGRT
SDMRTEIIRMMESARPEDVSFQGRGVFELSDEKAASPIVPSFDMSNEGSY
FFGDNAEEYDN

### Example 9

### Construction of NP-8×(M2e) (C8-non-his tagged)

A construct containing 8 copies of the M2e gene fused 3′ to the nucleoprotein gene is made and expressed in E. coli. The following is the protein sequence of the fusion protein:

(SEQ ID NO: 18)

MASQGTKRSYEQMETDGERQNATEIRASVGKMIGGIGRFYIQMGTELKLS
DYEGRLIQNSLTIERMVLSAFDERRNKYLEEHPSAGKDPKKTGGPIYRRV
NGKWMRELILYDKEEIRRIWRQANNGDDATAGLTHMMIWHSNLNDATYQR
TRALVRTGMDPRMCSLMQGSTLPRRSGAAGAAVKGVGTMVMELVRMIKRG
INDRNFWRGENGRKTRIAYERMCNILKGKFQTAAQKAMMDQVRESRNPGN
AEFEDLTFLARSALILRGSVAHKSCLPACVYGPAVASGYDFEREGYSLVG
IDPFRLLQNSQVYSLIRPNENPAHKSQLVWMACHSAAFEDLRVLSFIKGT
KVLPRGKLSTRGVQIASNENMETMESSTLELRSRYWAIRTRSGGNTNQQR
ASAGQISIQPTFSVQRNLPFDRTTIMAAFNGNTEGRTSDMRTEIIRMMES
ARPEDVSFQGRGVFELSDEKAASPIVPSFDMSNEGSYFFGDNAEEYDNSL
LTEVETPIRNEWGSRSNDSSDSLLTEVETPIRNEWGSRSNDSSDSLLTEV
ETPIRNEWGSRSNDSSDSLLTEVETPIRNEWGSRSNDSSDSLLTEVETPI
RNEWGSRSNDSSDSLLTEVETPIRNEWGSRSNDSSDSLLTEVETPLRNEW
GSRSNDSSDSLLTEVETPIRNEWGSRSNDSSD

### Example 10

### Covalent and Non-covalent Conjugates of NP, M2e and IMC

This example describes various covalent and non-covalent conjugates comprising NP, M2e and IMC that were made. A “double conjugate” was made by conjugating acetylated M2e peptide to 3′thio 295 ISS. Multiple (including single) copies were then in turn conjugated to NP protein. A “competitive binding conjugate” NP protein was simultaneously conjugated with NHS-activated M2e peptide and NHS-activated 3′295 ISS. By adding all reactants simultaneously, the IMC and M2e peptide compete to bind to the same sites on the NP protein. An “ionic association conjugate” was made by using the native RNA-binding pocket in the NP protein to non-covalently capture the IMC component of M2e-IMC conjugates. An excess of M2e-IMC conjugate was reacted with free NP protein, resulting in a noncovalent protein-conjugated peptide complex.

### Example 11

### M2e Peptide Conjugated to IMC Induces Strong Antibody Responses when Delivered with Alum

Groups of 10 BALB/c mice were immunized by intramuscular injection twice at a two week interval with either a synthetic peptide representing the extracellular domain of the influenza M2 protein (M2e) alone (5 μg), M2e (5 ,g) mixed with 1018 ISS (20 μg), M2e (5 μg) conjugated to 1018 ISS (approximately 20 μg), or the M2e-1018 ISS conjugate bound to alum. Two weeks after the second immunization, mice were bled and anti-M2e peptide IgG1 and IgG2a antibody titers were measured by ELISA. M2e alone was not immunogenic and did not induce detectable IgG1 or IgG2a antibodies. Similarly, the M2e mixed with 1018 ISS was not immunogenic. The M2e-1018 ISS conjugate was immunogenic and induced anti-M2e geometric mean titers of approximately 21,000 and 10,000, respectively, for IgG1 and IgG2a. The M2e-1018 ISS conjugate delivered bound to alum was very immunogenic and induced anti-M2e titers of 94,000 and 39,500, respectively, for IgG1 and IgG2a.

### Example 12

### M2c-1018 ISS conjugate is Immunogenic when Delivered with Alum or DOTAP and Addition of NP Affects M2e Response

Groups of 10 BALB/c mice were immunized by intramuscular injection twice at a two week interval with either M2e (5 μg) conjugated to 1018 ISS (approximately 20 μg), or the M2e-l 01s 1SS conjugate mixed with influenza nucleoprotein (NP, 10 μg), the M2e-1018 ISS conjugate bound to alum, the M2e-1018 ISS conjugate bound to alum and mixed with NP, or the M2e-1018 ISS conjugate delivered with the cationic lipid DOTAP, or the M2e-1018 ISS conjugate mixed with NP and delivered with DOTAP. Two weeks after the second immunization, mice were bled and anti-M2e peptide IgG1 and IgG2a antibody titers were measured by ELISA. As in example 1, M2e-1018 ISS conjugate was immunogenic and induced relatively low anti-M2e geometric mean titers of approximately 6,600 and 2,000, respectively, for IgG1 and IgG2a. The M2e-1018 ISS conjugate mixed with NP gave reduced anti-M2e IgG1 titers (geometric mean of 1,000) but very similar IgG2a titers (2,200) compared to the M2e- 1018 ISS conjugate alone. Delivery of the M2e-IMC conjugate in a polymeric configuration on alum induced both anti-M2e IgG1 and IgG2a titers that were significantly higher than those induced with the M2e-1018 ISS conjugate alone (geometric mean of 21,000 and 14,000, respectively). Again, adding NP to the M2e-1018 ISS conjugate+alum formulation reduced the resulting anti-M2e IgG1 titers by about 50% and increased the resulting anti-M2e IgG2a titers by 2-fold. Delivering M2e-1018 ISS in the DOTAP formulation induced similar IgG1 titers to the M2e-1018 ISS+alum formulation but induced significantly less IgG2a response than did the alum formulation.

### Example 13

### Immunogenicity of M2e-1018 ISS Alum Formulations

Groups of 5 BALB/c mice were immunized by intramuscular injection twice at a two week interval with either M2e (5 μg) conjugated to 1018 ISS (approximately 20 μg) delivered with alum, M2e (5 μg) mixed with 1018 ISS (20 μg) delivered with alum, M2e (5 μg) mixed with 1018 ISS (20 μg) and NP (10 μg) delivered with alum, or the M2e-1018 ISS conjugate mixed with NP (10 μg) and alum. Two weeks after the second immunization, mice were bled and anti-M2e peptide IgG1 and IgG2a antibody titers were measured by ELISA. M2e-1018 ISS delivered with alum induced significantly higher anti-M2e IgG1 responses than did M2e mixed with 1018 ISS and delivered with alum (266,000 vs. 17,000 respectively). Addition of NP to the non-IMC conjugated M2e+alum dramatically reduced both IgG1 (17,000 to 700) and IgG2a (13,000 to <600) responses to M2e. Consistent with example 2, addition of NP to the M2e-1018 ISS conjugate+alum formulation decreased anti-M2e IgG1 responses slightly (187,000 vs. 266,000) and increased anti-M2e IgG2a responses about 2-fold (40,000 vs. 15,500).

### Example 14

### A Fusion Protein of M2e and NP can Induce Antibody Responses to both M2e and NP.

The fusion protein N8 (non-His-tagged) as described in Example 5 was constructed as described therein. Groups of 5 BALB/c mice were immunized by intramuscular injection twice at a two week interval with either N8 fusion protein alone (10 μg), N8 fusion protein (10 μg) delivered with Complete Freund's adjuvant (CFA) on the primary injection and Incomplete Freund's adjuvant (IFA) on the secondary immunization, or with M2e-1018 ISS conjugate (5 μg M2e peptide, 20 μg 1018 ISS) delivered with alum. Two weeks after the second immunization, mice were bled and anti-M2e peptide and anti-NP IgG1 and IgG2a antibody titers were measured by ELISA.

The N8 fusion protein alone generated low but measurable IgG1 and IgG2a responses to M2e (5,600 and 2,000 respectively). When the N8 fusion protein was delivered with CFA/IFA, anti-M2e IgG1 titers were increased about 17-fold (95,000) and IgG2a titers were increased about 4-fold (9,400) compared to antigen alone. The anti-M2e IgG1 titers induced with N8 M2e/NP were similar to the IgG1 titers generated with the M2e peptide-IMC conjugate+alum formulation, but the IgG2a titers were about 5-fold lower (118,000 and 48,000 respectively). The N8 M2e/NP fusion protein generated strong anti-NP IgG1 titers that were similar with or without the CFA/IFA adjuvant (104,000 and 110,000 respectively). Anti-NP IgG2a responses were similar for the N8 fusion protein with or without the CFA/IFA adjuvant and were about 6-fold lower than the IgG1 titers. As expected the M2e peptide-IMC conjugate+alum formulation generated no measurable antibody response to NP.

### Example 15

### Immunogenicity of M2e/NP Fusion Proteins with Different Adjuvants

The fusion proteins N8 (non-His-tagged) (as described in Example 5) and N4/C4 (non-His-tagged) (as described in Example 6) were constructed as described therein. Groups of 5 BALB/c mice were immunized by intramuscular injection twice at a two week interval with either N4/C4 fusion protein (10 μg) delivered with alum, N8 fusion protein (10 μg) delivered with alum, N4/C4 fusion protein (10 μg) delivered with Iscomatrix adjuvant, N8 fusion protein (10 μg) delivered with Iscomatrix, the N8 fusion protein (10 μg) mixed with 1018 ISS (10 μg) or with M2e peptide-1018 ISS conjugate (5 μg M2e peptide, 20 μg 1018 ISS) delivered with alum. Two weeks after the second immunization, mice were bled and anti-M2e peptide and anti-NP IgG1 and IgG2a antibody titers were measured by ELISA.

The N8 and N4/C4 fusion proteins delivered with alum or with Iscomatrix all produced similar anti-M2e IgG1 titers, and these titers were in the same range and titers generated with the M2e peptide-1018 ISS+alum formulation (45,000-56,000 vs. 74,500). The N8+1018 ISS formulation produced very low anti-M2e IgG1 titers (1,000). The fusion proteins delivered with Iscomatrix or 1018 ISS, and the M2e peptide-IMC+alum formulations all produced higher anti-M2e IgG2a titers than the fusion protein+alum formulations (4,200 to 19,000 vs. 1,500). This is consistent with the known ability of Iscomatrix and 1018 ISS adjuvants to induce a Th1 response leading to IgG2a production in the mouse.

The N8+alum and N4/C4+alum formulations both induced strong IgG1 responses and low IgG2a responses against NP (29,000 and 49,000 respectively). The N8+Iscomatrix and N4C4+Iscomatrix formulation both induced a more balanced IgG1/IgG2a response against NP than did the alum formulation (16,000 and 9,000 for IgG1, and 28,000 and 16,000 for IgG2a respectively). The N8+1018 ISS formulation induced low IgG1 and IgG2a responses against NP. The M2e peptide formulation did not induce measurable antibody responses against NP, as expected.

### Example 16

### Immunogenicity of M2e/NP Fusion Proteins Delivered with Different Adjuvants

Groups of 5 BALB/c mice were immunized by intramuscular injection twice at a two week interval with N8 fusion protein (25 μg) delivered alone, with alum, with MF59 adjuvant, with MF59+1018 ISS (25 μg), or with 1018 ISS (25 μg), or with N4/C4 fusion protein (25 μg) delivered with alum. A control group of 5 mice received only PBS. Two weeks after the second immunization, mice were bled and anti-M2e peptide and anti-NP IgG1 and IgG2a antibody titers were measured by ELISA. Four weeks after the second immunization, mice were sacrificed, spleens were harvested and spleen cells were used in an ELISPOT assay to determine the number of NP-specific T cells producing IFNγ.

The N8 fusion protein alone produced low levels of both M2e-specific IgG1 and IgG2a antibodies (2,700 and <600 respectively). N8 fusion protein delivered with alum, N8 delivered with MF59, and N4/C4 delivered with alum all produced similar anti-M2e antibody titers that were dominated by IgG1 over IgG2a (33,000, 21,500 and 40,000 for IgG1 vs. <600, 800 and 1000 for IgG2a respectively). Including 1018 ISS in the N8+MF59 formulation reduced the anti-M2e IgG1 titers by about 50% but increased IgG2a titers by 28-fold (10,000 vs. 21,000 respectively). The N8+1018 ISS formulation produced low anti-M2e titers for both IgG1 and IgG2a(900 and 2,400).

N8 fusion alone produced strong anti-NP titers that were dominated by IgG1 over IgG2a (115,000 and 9,000 respectively). Using alum or MF59 adjuvants increased these responses about 2-fold. N4/C4+alum produced anti-NP titers that were similar to those produced with N8+alum. Delivery of N8 with MF59+1018 ISS induced a shift in antibody response resulting in very high IgG2a responses and much lower IgG1 responses than the alum or MF59 formulations (413,000 and 49,000 respectively). Delivery of N8 with 1018 showed a shift from IgG1 to IgG2a (2,600 and 40,000 respectively), but overall titers were much lower than those in the N8+MF59+1018 ISS group.

Using the ELISPOT assay, the N8 alone, N8+alum, N8+MF59 and N8+1018 ISS formulations all produced similar numbers of IFNγ spot forming cells after restimulation with an NP-specific CD8 peptide for BALB/c mice or with a peptide pool covering the entire NP amino acid sequence (60-90 sfu per 10⁶ cells). The number of NP specific IFNγ spot forming cells was substantially higher in the group receiving C8+MF59+1018 ISS than in the other groups (180-290 sfu per 10⁶ cells).

### Example 17

### Animal Studies

BALB/c mice (10 per group) are immunized twice (e.g., at week 0 and 2) with the NP/M2c constructs shown above, the NP/M2c constructs conjugated to an IMC or control materials (NP and M2e alone, NP-IMC, and M2e-IMC/alum). Two weeks post second immunization, the mice are bled and serum is assayed to determine NP and M2e-specific antibody responses. The mice spleens are harvested and splenocytes are assayed in vitro for NP-specific cell mediated immune responses using IFN-γ and IL-4 ELISPOT, and/or cytokine ELISA.

### Example 18

### Immunization with Multimers and TIV

Individuals who are at risk for infection with influenza or who are in need of inducement of immune responses are vaccinated with a combination of one or more of the following: (1) M2e/IMC multimer+trivalent inactivated vaccine (TIV); (2) M2e/IMC and NP/IMC multimers+TIV; (3) M2e/NP/IMC multimers+TIV. The individual can be optionally monitored either before or after the vaccination to determine the immunological responses (e.g., humoral and/or cellular responses) and/or physiological responses (e.g., lessening of symptoms associates with influenza infection). The amount of multimers used is between 1 μg and 100 μg and is used in combination with TIV for reducing the risk of infection with influenza virus.

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention.

All patents, patent applications, and publications cited herein are hereby incorporated by reference in their entirety for all purposes to the same extent as if each individual publication, patent or patent application were specifically and individually indicated to be so incorporated by reference.

1. A composition comprising a multimer of an extracellular domain of influenza matrix protein (M2c) wherein the M2c is presented to the immune system as a multimeric display and is capable of inducing an immune response in an individual.

2. The composition of claim 1 wherein the multimeric display is accomplished by associating at least two copies of M2e with a non-protein platform molecule.

3. The composition of claim 1 further comprising an immunomodulatory compound (IMC) comprising an immunostimulatory sequence (ISS).

4. The composition of claim 3 wherein the IMC is associated with the multimer.

5. The composition of claim 4 where the IMC is covalently linked to the multimer.

6. The composition of claim 1 further comprising nucleoprotein (NP).

7. The composition of claim 6 wherein the multimeric display is accomplished by linking at least two copies of M2e covalently or ionically to NP.

8. The composition of claim 7 wherein the multimeric display is accomplished by a fusion protein comprising at least two copies of M2e and NP.

9. The composition of claim 8 wherein the copies of M2e are situated on the carboxy terminus side of NP.

10. The composition of claim 9 wherein the fusion protein comprises eight copies of M2e on the carboxy terminus side of NP.

11. The composition of claim 8 wherein the copies of M2e are situated on the amino terminus side of NP.

12. The composition of claim 11 wherein the fusion protein comprises eight copies of M2e on the amino terminus side of NP.

13. The composition of claim 8 wherein copies of M2e are situated on both the amino and carboxy termini sides of NP.

14. The composition of claim 13 wherein the fusion protein comprises four copies of M2e on the amino terminus side of NP and four copies of M2e on the carboxy terminus side of NP.

15. The composition of claim 6 further comprising an IMC comprising an ISS associated with NP.

16. The composition of claim 7 further comprising an IMC comprising an ISS associated with NP.

17. The composition of claim 8 further comprising an IMC comprising an ISS associated with NP.

18. A composition comprising NP covalently linked to an IMC comprising an ISS.

19. The composition of claim 8 further comprising a carrier selected from the group consisting of alum, microparticles, liposomes, and nanoparticles.

20. A vaccine comprising a composition comprising a multimer of an extracellular domain of influenza matrix protein (M2e) wherein the M2e is presented to the immune system as a multimeric display and is capable of inducing an immune response in an individual.

21. The vaccine of claim 20 further comprising an adjuvant.

22. The vaccine of claim 20 further comprising one or more components of at least one trivalent inactivated influenza vaccine (TIV).

23. The vaccine of claim 20 wherein the multimeric display is accomplished by a fusion protein comprising at least two copies of M2e fused to NP.

24. The vaccine of claim 23 further comprising an adjuvant.

25. The vaccine of claim 23 further comprising one or more components of at least one trivalent inactivated influenza vaccine (TIV).

26. A method for ameliorating one or more symptoms associated with infection with influenza virus in an individual by administering to the individual the vaccine of claim 23.

27. A method for reducing the likelihood of infection with influenza virus in an individual comprising administering to the individual the vaccine of claim 23.

28. A method for reducing the likelihood of infection with influenza virus in an individual comprising administering to the individual the vaccine of claim 23 and one or more components of monovalent inactivated vaccine.


SEQUENCE LISTING (79 sequences)

SEQ ID NO: 1 - DNA, 2082 bp, Artificial Sequence
  Synthetic Construct
catatgtctctgttaacggaagtcgagacacccatccggaatgagtggggttcccgtagtaatgatagttcggatagcttactgaccgaggttgaaacacctattcgtaacgaatggggtagccggtcaaatgactcgagcgattcgttgttgaccgaagtagagaccccaatccgcaatgaatggggctcccggagtaacgatagcagcgactccttactgacggaggtggaaacgcccatccgtaacgagtggggttctagaagtaacgattcctcggatagcttattaacagaagtcgaaacgcctattcgcaatgaatggggttcgcgttcgaatgattccagtgatagcctgttaacggaagttgaaactccgatccgtaatgagtggggcagccgtagcaacgactcgagcgactccctgctcactgaggttgagacaccaatccggaacgaatggggctcgcgctcgaacgattcttccgattctctgctgaccgaagtagaaactcctattcgtaatgaatggggttcccgttccaatgatagcagcgatatggcttcccagggtactaaacgtagctatgaacagatggaaaccgatggtgaacgtcagaacgcgactgaaatccgtgctagcgtaggtaaaatgatcggtggtatcggtcgtttctacatccagatgtgcactgaacttaaacttagcgactatgaaggtcgtctgatccagaattctctgaccattgaacgtatggttcttagcgcgtttgatgaacgtcgtaacaaataccttgaagaacacccgtctgctggtaaagaccctaaaaaaactggtggtccgatctatcgtcgtgttaacggtaaatggatgcgtgaactgatcctgtatgacaaagaagaaatccgtcgtatttggagacaggctaacaatggtgatgacgcgaccgctggactgacccacatgatgatttggcacagcaacctgaacgatgcgacctaccagcgtacccgtgcgttagtacgtaccggtatggacccgcgtatgtgtagcctgatgcaaggtagcactctgcctcgtcgttctggtgcggctggtgcggcggttaaaggtgtgggtactatggttatggaactggttcgtatgattaaacgtggtatcaacgatcgtaacttttggcgtggtgaaaatggtcgtaaaacccgtatcgcgtatgaacgtatgtgcaacatccttaaaggtaaatttcagaccgcagcgcagaaagctatgatggaccaggttcgtgaatctcgtaatccgggtaatgctgagttcgaagacctgaccttcctggctcgttctgcactgatcctgcgtggtagcgtagcgcacaaatcttgcctgccagcgtgtgtttacggtccggcggttgctagcggttatgacttcgaacgtgaaggttactctttggttggtattgacccgttccgactgctccagaactcccaggtttactctctgatccgtcctaacgaaaacccggcgcataaatctcagttagtttggatggcttgtcactctgcggcgtttgaagacctgcgtgttctgagcttcattaaaggtactaaagttctgccgcgtggtaaactgtctacccgtggtgttcagatcgctagcaatgaaaacatggaaactatggaatctagcaccctagaactgcgtagtcgttattgggcgatccgtacccgtagcggtggtaataccaaccagcagcgtgcgagcgcgggtcagattagcatccagccgacctttagcgttcagcgtaacctgccgtttgaccgtaccaccatcatggctgcgtttaacggtaacactgaaggtcgtaccagtgacatgcgtactgaaatcatccgtatgatggaatctgctcgaccggaagacgtgagctttcagggtcgtggtgtttttgaacttagcgatgaaaaagctgctagcccgatcgttcctagctttgacatgtctaacgaaggtagctacttcttcggtgacaacgctgaggaatatgacaaccatcatcaccatcaccattaataaggatcc

SEQ ID NO: 2 - protein, 689 aa, Artificial Sequence
  Synthetic Construct
MSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDMASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDNHHHHHH

SEQ ID NO: 3 - DNA, 2082 bp, Artificial Sequence
  Synthetic Construct
catatgagcctgttaaccgaagtcgagacgcctattcgtaatgaatggggcagtcggtcgaacgatagctcggatagcctgctgacggaggtggaaaccccgatccgtaacgagtggggctctcgtagtaacgactcgagcgatagcttactgactgaagttgaaactccaattcgcaatgagtggggtagccgcagcaatgatagcagtgatagcttattaacggaagttgaaacgcctatccggaacgaatggggttctagaagcaacgatagtagcgatatggcttcccagggtactaaacgtagctatgaacagatggaaaccgatggtgaacgtcagaacgcgactgaaatccgtgctagcgtaggtaaaatgatcggtggtatcggtcgtttctacatccagatgtgcactgaacttaaacttagcgactatgaaggtcgtctgatccagaattctctgaccattgaacgtatggttcttagcgcgtttgatgaacgtcgtaacaaataccttgaagaacacccgtctgctggtaaagaccctaaaaaaactggtggtccgatctatcgtcgtgttaacggtaaatggatgcgtgaactgatcctgtatgacaaagaagaaatccgtcgtatttggagacaggctaacaatggtgatgacgcgaccgctggactgacccacatgatgatttggcacagcaacctgaacgatgcgacctaccagcgtacccgtgcgttagtacgtaccggtatggacccgcgtatgtgtagcctgatgcaaggtagcactctgcctcgtcgttctggtgcggctggtgcggcggttaaaggtgtgggtactatggttatggaactggttcgtatgattaaacgtggtatcaacgatcgtaacttttggcgtggtgaaaatggtcgtaaaacccgtatcgcgtatgaacgtatgtgcaacatccttaaaggtaaatttcagaccgcagcgcagaaagctatgatggaccaggttcgtgaatctcgtaatccgggtaatgctgagttcgaagacctgaccttcctggctcgttctgcactgatcctgcgtggtagcgtagcgcacaaatcttgcctgccagcgtgtgtttacggtccggcggttgctagcggttatgacttcgaacgtgaaggttactctttggttggtattgacccgttccgactgctccagaactcccaggtttactctctgatccgtcctaacgaaaacccggcgcataaatctcagttagtttggatggcttgtcactctgcggcgtttgaagacctgcgtgttctgagcttcattaaaggtactaaagttctgccgcgtggtaaactgtctacccgtggtgttcagatcgctagcaatgaaaacatggaaactatggaatctagcaccctagaactgcgtagtcgttattgggcgatccgtacccgtagcggtggtaataccaaccagcagcgtgcgagcgcgggtcagattagcatccagccgacctttagcgttcagcgtaacctgccgtttgaccgtaccaccatcatggctgcgtttaacggtaacactgaaggtcgtaccagtgacatgcgtactgaaatcatccgtatgatggaatctgctcgaccggaagacgtgagctttcagggtcgtggtgtttttgaacttagcgatgaaaaagctgctagcccgatcgttcctagctttgacatgtctaacgaaggtagctacttcttcggtgacaacgctgaggaatatgacaactctctgttgactgaagtagagactccaattcgtaacgaatggggtagccgttctaacgactcttccgactctctgctcaccgaggttgaaaccccgattcgcaatgaatggggctcgcgttccaatgactcgagcgattctctcctgacggaggttgagacgcctatccgtaatgagtggggttcccggagcaatgattcttctgattctctgctgactgaagtcgaaaccccgattcggaacgagtggggcagtcgttcaaatgactcgtcggaccatcatcatcaccatcattaataaggatcc

SEQ ID NO: 4 - protein, 689 aa, Artificial Sequence
  Synthetic Construct
MSLLTEVETPIRNEWGSRSNDSSDSLLTEVETPIRNEWGSRSNDSSDSLLTEVETPIRNEWGSRSNDSSDSLLTEVETPIRNEWGSRSNDSSDMASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDNSLLTEVETPIRNEWGSRSNDSSDSLLTEVETPIRNEWGSRSNDSSDSLLTEVETPIRNEWGSRSNDSSDSLLTEVETPIRNEWGSRSNDSSDHHHHHH

SEQ ID NO: 5 - DNA, 1806 bp, Artificial Sequence
  Synthetic Construct
catatgagcctgttaacggaggtggaaactccaattcggaatgaatggggttcgcgcagcaatgatagctcggatagcttactgaccgaagtcgaaacacccatccgtaacgaatggggcagccgtagcaacgactcgagcgactccctgctcactgaggttgagaccccgatccgcaatgagtggggctcgcgctcgaacgattcttccgattctctgctgaccgaagtagaaactcctattcgtaatgaatggggttcccgttccaatgatagcagcgatatggcttcccagggtactaaacgtagctatgaacagatggaaaccgatggtgaacgtcagaacgcgactgaaatccgtgctagcgtaggtaaaatgatcggtggtatcggtcgtttctacatccagatgtgcactgaacttaaacttagcgactatgaaggtcgtctgatccagaattctctgaccattgaacgtatggttcttagcgcgtttgatgaacgtcgtaacaaataccttgaagaacacccgtctgctggtaaagaccctaaaaaaactggtggtccgatctatcgtcgtgttaacggtaaatggatgcgtgaactgatcctgtatgacaaagaagaaatccgtcgtatttggagacaggctaacaatggtgatgacgcgaccgctggactgacccacatgatgatttggcacagcaacctgaacgatgcgacctaccagcgtacccgtgcgttagtacgtaccggtatggacccgcgtatgtgtagcctgatgcaaggtagcactctgcctcgtcgttctggtgcggctggtgcggcggttaaaggtgtgggtactatggttatggaactggttcgtatgattaaacgtggtatcaacgatcgtaacttttggcgtggtgaaaatggtcgtaaaacccgtatcgcgtatgaacgtatgtgcaacatccttaaaggtaaatttcagaccgcagcgcagaaagctatgatggaccaggttcgtgaatctcgtaatccgggtaatgctgagttcgaagacctgaccttcctggctcgttctgcactgatcctgcgtggtagcgtagcgcacaaatcttgcctgccagcgtgtgtttacggtccggcggttgctagcggttatgacttcgaacgtgaaggttactctttggttggtattgacccgttccgactgctccagaactcccaggtttactctctgatccgtcctaacgaaaacccggcgcataaatctcagttagtttggatggcttgtcactctgcggcgtttgaagacctgcgtgttctgagcttcattaaaggtactaaagttctgccgcgtggtaaactgtctacccgtggtgttcagatcgctagcaatgaaaacatggaaactatggaatctagcaccctagaactgcgtagtcgttattgggcgatccgtacccgtagcggtggtaataccaaccagcagcgtgcgagcgcgggtcagattagcatccagccgacctttagcgttcagcgtaacctgccgtttgaccgtaccaccatcatggctgcgtttaacggtaacactgaaggtcgtaccagtgacatgcgtactgaaatcatccgtatgatggaatctgctcgaccggaagacgtgagctttcagggtcgtggtgtttttgaacttagcgatgaaaaagctgctagcccgatcgttcctagctttgacatgtctaacgaaggtagctacttcttcggtgacaacgctgaggaatatgacaaccatcatcaccatcaccattaataaggatcc

SEQ ID NO: 6 - protein, 597 aa, Artificial Sequence
  Synthetic Construct
MSLLTEVETPIRNEWGSRSNDSSDSLLTEVETPIRNEWGSRSNDSSDSLLTEVETPIRNEWGSRSNDSSDSLLTEVETPIRNEWGSRSNDSSDMASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDNHHHHHH

SEQ ID NO: 7 - DNA, 1866 bp, Artificial Sequence
  Synthetic Construct
catatgtccctgctgacggaagtagaaaccccaattcgcaatgaatggggcagccgtagcaatgactcttctgacggttctgcgagcggtagcttgcttactgaagttgaaactcctatccgtaacgaatggggttcccgttctaacgactcgagcgacggcagcgcgtccggttctctgctgactgaggtcgagactccgattcgtaatgagtggggtagccgcagcaacgattcttccgatggctctgcttctggttccttgttgaccgaagttgaaacccctatccgcaacgaatggggctctcgctctaatgatagctctgatggttcggcttccggcatggcttcccagggtactaaacgtagctatgaacagatggaaaccgatggtgaacgtcagaacgcgactgaaatccgtgctagcgtaggtaaaatgatcggtggtatcggtcgtttctacatccagatgtgcactgaacttaaacttagcgactatgaaggtcgtctgatccagaattctctgaccattgaacgtatggttcttagcgcgtttgatgaacgtcgtaacaaataccttgaagaacacccgtctgctggtaaagaccctaaaaaaactggtggtccgatctatcgtcgtgttaacggtaaatggatgcgtgaactgatcctgtatgacaaagaagaaatccgtcgtatttggagacaggctaacaatggtgatgacgcgaccgctggactgacccacatgatgatttggcacagcaacctgaacgatgcgacctaccagcgtacccgtgcgttagtacgtaccggtatggacccgcgtatgtgtagcctgatgcaaggtagcactctgcctcgtcgttctggtgcggctggtgcggcggttaaaggtgtgggtactatggttatggaactggttcgtatgattaaacgtggtatcaacgatcgtaacttttggcgtggtgaaaatggtcgtaaaacccgtatcgcgtatgaacgtatgtgcaacatccttaaaggtaaatttcagaccgcagcgcagaaagctatgatggaccaggttcgtgaatctcgtaatccgggtaatgctgagttcgaagacctgaccttcctggctcgttctgcactgatcctgcgtggtagcgtagcgcacaaatcttgcctgccagcgtgtgtttacggtccggcggttgctagcggttatgacttcgaacgtgaaggttactctttggttggtattgacccgttccgactgctccagaactcccaggtttactctctgatccgtcctaacgaaaacccggcgcataaatctcagttagtttggatggcttgtcactctgcggcgtttgaagacctgcgtgttctgagcttcattaaaggtactaaagttctgccgcgtggtaaactgtctacccgtggtgttcagatcgctagcaatgaaaacatggaaactatggaatctagcaccctagaactgcgtagtcgttattgggcgatccgtacccgtagcggtggtaataccaaccagcagcgtgcgagcgcgggtcagattagcatccagccgacctttagcgttcagcgtaacctgccgtttgaccgtaccaccatcatggctgcgtttaacggtaacactgaaggtcgtaccagtgacatgcgtactgaaatcatccgtatgatggaatctgctcgaccggaagacgtgagctttcagggtcgtggtgtttttgaacttagcgatgaaaaagctgctagcccgatcgttcctagctttgacatgtctaacgaaggtagctacttcttcggtgacaacgctgaggaatatgacaaccatcaccatcatcaccactaataaggatcc

SEQ ID NO: 8 - protein, 617 aa, Artificial Sequence
  Synthetic Construct
MSLLTEVETPIRNEWGSRSNDSSDGSASGSLLTEVETPIRNEWGSRSNDSSDGSASGSLLTEVETPIRNEWGSRSNDSSDGSASGSLLTEVETPIRNEWGSRSNDSSDGSASGMASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDNHHHHHH

SEQ ID NO: 9 - DNA, 2064 bp, Artificial Sequence
  Synthetic Construct
catatgtctctgttaacggaagtcgagacacccatccggaatgagtggggttcccgtagtaatgatagttcggatagcttactgaccgaggttgaaacacctattcgtaacgaatggggtagccggtcaaatgactcgagcgattcgttgttgaccgaagtagagaccccaatccgcaatgaatggggctcccggagtaacgatagcagcgactccttactgacggaggtggaaacgcccatccgtaacgagtggggttctagaagtaacgattcctcggatagcttattaacagaagtcgaaacgcctattcgcaatgaatggggttcgcgttcgaatgattccagtgatagcctgttaacggaagttgaaactccgatccgtaatgagtggggcagccgtagcaacgactcgagcgactccctgctcactgaggttgagacaccaatccggaacgaatggggctcgcgctcgaacgattcttccgattctctgctgaccgaagtagaaactcctattcgtaatgaatggggttcccgttccaatgatagcagcgatatggcttcccagggtactaaacgtagctatgaacagatggaaaccgatggtgaacgtcagaacgcgactgaaatccgtgctagcgtaggtaaaatgatcggtggtatcggtcgtttctacatccagatgtgcactgaacttaaacttagcgactatgaaggtcgtctgatccagaattctctgaccattgaacgtatggttcttagcgcgtttgatgaacgtcgtaacaaataccttgaagaacacccgtctgctggtaaagaccctaaaaaaactggtggtccgatctatcgtcgtgttaacggtaaatggatgcgtgaactgatcctgtatgacaaagaagaaatccgtcgtatttggagacaggctaacaatggtgatgacgcgaccgctggactgacccacatgatgatttggcacagcaacctgaacgatgcgacctaccagcgtacccgtgcgttagtacgtaccggtatggacccgcgtatgtgtagcctgatgcaaggtagcactctgcctcgtcgttctggtgcggctggtgcggcggttaaaggtgtgggtactatggttatggaactggttcgtatgattaaacgtggtatcaacgatcgtaacttttggcgtggtgaaaatggtcgtaaaacccgtatcgcgtatgaacgtatgtgcaacatccttaaaggtaaatttcagaccgcagcgcagaaagctatgatggaccaggttcgtgaatctcgtaatccgggtaatgctgagttcgaagacctgaccttcctggctcgttctgcactgatcctgcgtggtagcgtagcgcacaaatcttgcctgccagcgtgtgtttacggtccggcggttgctagcggttatgacttcgaacgtgaaggttactctttggttggtattgacccgttccgactgctccagaactcccaggtttactctctgatccgtcctaacgaaaacccggcgcataaatctcagttagtttggatggcttgtcactctgcggcgtttgaagacctgcgtgttctgagcttcattaaaggtactaaagttctgccgcgtggtaaactgtctacccgtggtgttcagatcgctagcaatgaaaacatggaaactatggaatctagcaccctagaactgcgtagtcgttattgggcgatccgtacccgtagcggtggtaataccaaccagcagcgtgcgagcgcgggtcagattagcatccagccgacctttagcgttcagcgtaacctgccgtttgaccgtaccaccatcatggctgcgtttaacggtaacactgaaggtcgtaccagtgacatgcgtactgaaatcatccgtatgatggaatctgctcgaccggaagacgtgagctttcagggtcgtggtgtttttgaacttagcgatgaaaaagctgctagcccgatcgttcctagctttgacatgtctaacgaaggtagctacttcttcggtgacaacgctgaggaatatgacaactaataaggatcc

SEQ ID NO: 10 - protein, 683 aa, Artificial Sequence
  Synthetic Construct
MSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDMASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 11 - DNA, 2064 bp, Artificial Sequence
  Synthetic Construct
catatgagcctgttaaccgaagtcgagacgcctattcgtaatgaatggggcagtcggtcgaacgatagctcggatagcctgctgacggaggtggaaaccccgatccgtaacgagtggggctctcgtagtaacgactcgagcgatagcttactgactgaagttgaaactccaattcgcaatgagtggggtagccgcagcaatgatagcagtgatagcttattaacggaagttgaaacgcctatccggaacgaatggggttctagaagcaacgatagtagcgatatggcttcccagggtactaaacgtagctatgaacagatggaaaccgatggtgaacgtcagaacgcgactgaaatccgtgctagcgtaggtaaaatgatcggtggtatcggtcgtttctacatccagatgtgcactgaacttaaacttagcgactatgaaggtcgtctgatccagaattctctgaccattgaacgtatggttcttagcgcgtttgatgaacgtcgtaacaaataccttgaagaacacccgtctgctggtaaagaccctaaaaaaactggtggtccgatctatcgtcgtgttaacggtaaatggatgcgtgaactgatcctgtatgacaaagaagaaatccgtcgtatttggagacaggctaacaatggtgatgacgcgaccgctggactgacccacatgatgatttggcacagcaacctgaacgatgcgacctaccagcgtacccgtgcgttagtacgtaccggtatggacccgcgtatgtgtagcctgatgcaaggtagcactctgcctcgtcgttctggtgcggctggtgcggcggttaaaggtgtgggtactatggttatggaactggttcgtatgattaaacgtggtatcaacgatcgtaacttttggcgtggtgaaaatggtcgtaaaacccgtatcgcgtatgaacgtatgtgcaacatccttaaaggtaaatttcagaccgcagcgcagaaagctatgatggaccaggttcgtgaatctcgtaatccgggtaatgctgagttcgaagacctgaccttcctggctcgttctgcactgatcctgcgtggtagcgtagcgcacaaatcttgcctgccagcgtgtgtttacggtccggcggttgctagcggttatgacttcgaacgtgaaggttactctttggttggtattgacccgttccgactgctccagaactcccaggtttactctctgatccgtcctaacgaaaacccggcgcataaatctcagttagtttggatggcttgtcactctgcggcgtttgaagacctgcgtgttctgagcttcattaaaggtactaaagttctgccgcgtggtaaactgtctacccgtggtgttcagatcgctagcaatgaaaacatggaaactatggaatctagcaccctagaactgcgtagtcgttattgggcgatccgtacccgtagcggtggtaataccaaccagcagcgtgcgagcgcgggtcagattagcatccagccgacctttagcgttcagcgtaacctgccgtttgaccgtaccaccatcatggctgcgtttaacggtaacactgaaggtcgtaccagtgacatgcgtactgaaatcatccgtatgatggaatctgctcgaccggaagacgtgagctttcagggtcgtggtgtttttgaacttagcgatgaaaaagctgctagcccgatcgttcctagctttgacatgtctaacgaaggtagctacttcttcggtgacaacgctgaggaatatgacaactctctgttgactgaagtagagactccaattcgtaacgaatggggtagccgttctaacgactcttccgactctctgctcaccgaggttgaaaccccgattcgcaatgaatggggctcgcgttccaatgactcgagcgattctctcctgacggaggttgagacgcctatccgtaatgagtggggttcccggagcaatgattcttctgattctctgctgactgaagtcgaaaccccgattcggaacgagtggggcagtcgttcaaatgactcgtcggactaataaggatcc

SEQ ID NO: 12 - protein, 683 aa, Artificial Sequence
  Synthetic Construct
MSLLTEVETPIRNEWGSRSNDSSDSLLTEVETPIRNEWGSRSNDSSDSLLTEVETPIRNEWGSRSNDSSDSLLTEVETPIRNEWGSRSNDSSDMASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDNSLLTEVETPIRNEWGSRSNDSSDSLLTEVETPIRNEWGSRSNDSSDSLLTEVETPIRNEWGSRSNDSSDSLLTEVETPIRNEWGSRSNDSSD

SEQ ID NO: 13 - DNA, 1788 bp, Artificial Sequence
  Synthetic Construct
catatgagcctgttaacggaggtggaaactccaattcggaatgaatggggttcgcgcagcaatgatagctcggatagcttactgaccgaagtcgaaacacccatccgtaacgaatggggcagccgtagcaacgactcgagcgactccctgctcactgaggttgagaccccgatccgcaatgagtggggctcgcgctcgaacgattcttccgattctctgctgaccgaagtagaaactcctattcgtaatgaatggggttcccgttccaatgatagcagcgatatggcttcccagggtactaaacgtagctatgaacagatggaaaccgatggtgaacgtcagaacgcgactgaaatccgtgctagcgtaggtaaaatgatcggtggtatcggtcgtttctacatccagatgtgcactgaacttaaacttagcgactatgaaggtcgtctgatccagaattctctgaccattgaacgtatggttcttagcgcgtttgatgaacgtcgtaacaaataccttgaagaacacccgtctgctggtaaagaccctaaaaaaactggtggtccgatctatcgtcgtgttaacggtaaatggatgcgtgaactgatcctgtatgacaaagaagaaatccgtcgtatttggagacaggctaacaatggtgatgacgcgaccgctggactgacccacatgatgatttggcacagcaacctgaacgatgcgacctaccagcgtacccgtgcgttagtacgtaccggtatggacccgcgtatgtgtagcctgatgcaaggtagcactctgcctcgtcgttctggtgcggctggtgcggcggttaaaggtgtgggtactatggttatggaactggttcgtatgattaaacgtggtatcaacgatcgtaacttttggcgtggtgaaaatggtcgtaaaacccgtatcgcgtatgaacgtatgtgcaacatccttaaaggtaaatttcagaccgcagcgcagaaagctatgatggaccaggttcgtgaatctcgtaatccgggtaatgctgagttcgaagacctgaccttcctggctcgttctgcactgatcctgcgtggtagcgtagcgcacaaatcttgcctgccagcgtgtgtttacggtccggcggttgctagcggttatgacttcgaacgtgaaggttactctttggttggtattgacccgttccgactgctccagaactcccaggtttactctctgatccgtcctaacgaaaacccggcgcataaatctcagttagtttggatggcttgtcactctgcggcgtttgaagacctgcgtgttctgagcttcattaaaggtactaaagttctgccgcgtggtaaactgtctacccgtggtgttcagatcgctagcaatgaaaacatggaaactatggaatctagcaccctagaactgcgtagtcgttattgggcgatccgtacccgtagcggtggtaataccaaccagcagcgtgcgagcgcgggtcagattagcatccagccgacctttagcgttcagcgtaacctgccgtttgaccgtaccaccatcatggctgcgtttaacggtaacactgaaggtcgtaccagtgacatgcgtactgaaatcatccgtatgatggaatctgctcgaccggaagacgtgagctttcagggtcgtggtgtttttgaacttagcgatgaaaaagctgctagcccgatcgttcctagctttgacatgtctaacgaaggtagctacttcttcggtgacaacgctgaggaatatgacaactaataaggatcc

SEQ ID NO: 14 - protein, 591 aa, Artificial Sequence
  Synthetic Construct
MSLLTEVETPIRNEWGSRSNDSSDSLLTEVETPIRNEWGSRSNDSSDSLLTEVETPIRNEWGSRSNDSSDSLLTEVETPIRNEWGSRSNDSSDMASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 15 - DNA, 1848 bp, Artificial Sequence
  Synthetic Construct
catatgtccctgctgacggaagtagaaaccccaattcgcaatgaatggggcagccgtagcaatgactcttctgacggttctgcgagcggtagcttgcttactgaagttgaaactcctatccgtaacgaatggggttcccgttctaacgactcgagcgacggcagcgcgtccggttctctgctgactgaggtcgagactccgattcgtaatgagtggggtagccgcagcaacgattcttccgatggctctgcttctggttccttgttgaccgaagttgaaacccctatccgcaacgaatggggctctcgctctaatgatagctctgatggttcggcttccggcatggcttcccagggtactaaacgtagctatgaacagatggaaaccgatggtgaacgtcagaacgcgactgaaatccgtgctagcgtaggtaaaatgatcggtggtatcggtcgtttctacatccagatgtgcactgaacttaaacttagcgactatgaaggtcgtctgatccagaattctctgaccattgaacgtatggttcttagcgcgtttgatgaacgtcgtaacaaataccttgaagaacacccgtctgctggtaaagaccctaaaaaaactggtggtccgatctatcgtcgtgttaacggtaaatggatgcgtgaactgatcctgtatgacaaagaagaaatccgtcgtatttggagacaggctaacaatggtgatgacgcgaccgctggactgacccacatgatgatttggcacagcaacctgaacgatgcgacctaccagcgtacccgtgcgttagtacgtaccggtatggacccgcgtatgtgtagcctgatgcaaggtagcactctgcctcgtcgttctggtgcggctggtgcggcggttaaaggtgtgggtactatggttatggaactggttcgtatgattaaacgtggtatcaacgatcgtaacttttggcgtggtgaaaatggtcgtaaaacccgtatcgcgtatgaacgtatgtgcaacatccttaaaggtaaatttcagaccgcagcgcagaaagctatgatggaccaggttcgtgaatctcgtaatccgggtaatgctgagttcgaagacctgaccttcctggctcgttctgcactgatcctgcgtggtagcgtagcgcacaaatcttgcctgccagcgtgtgtttacggtccggcggttgctagcggttatgacttcgaacgtgaaggttactctttggttggtattgacccgttccgactgctccagaactcccaggtttactctctgatccgtcctaacgaaaacccggcgcataaatctcagttagtttggatggcttgtcactctgcggcgtttgaagacctgcgtgttctgagcttcattaaaggtactaaagttctgccgcgtggtaaactgtctacccgtggtgttcagatcgctagcaatgaaaacatggaaactatggaatctagcaccctagaactgcgtagtcgttattgggcgatccgtacccgtagcggtggtaataccaaccagcagcgtgcgagcgcgggtcagattagcatccagccgacctttagcgttcagcgtaacctgccgtttgaccgtaccaccatcatggctgcgtttaacggtaacactgaaggtcgtaccagtgacatgcgtactgaaatcatccgtatgatggaatctgctcgaccggaagacgtgagctttcagggtcgtggtgtttttgaacttagcgatgaaaaagctgctagcccgatcgttcctagctttgacatgtctaacgaaggtagctacttcttcggtgacaacgctgaggaatatgacaactaataaggatcc

SEQ ID NO: 16 - protein, 611 aa, Artificial Sequence
  Synthetic Construct
MSLLTEVETPIRNEWGSRSNDSSDGSASGSLLTEVETPIRNEWGSRSNDSSDGSASGSLLTEVETPIRNEWGSRSNDSSDGSASGSLLTEVETPIRNEWGSRSNDSSDGSASGMASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 17 - protein, 23 aa, Influenza virus
SLLTEVETPIRNGWECRCNDSSD

SEQ ID NO: 18 - protein, 682 aa, Artificial Sequence
  Synthetic Construct
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDNSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGSRSNDSSD

SEQ ID NO: 19 - protein, 23 aa, Influenza virus
SLLTEVETPIRNEWGCRCNDSSD

SEQ ID NO: 20 - protein, 23 aa, Influenza virus
SLLTEVETPTRNGWECKCSDSSD

SEQ ID NO: 21 - DNA, 22 bp, Influenza virus
tgactgtgaacgttcgagatga

SEQ ID NO: 22 - protein, 23 aa, Influenza virus
SLLTEVETPIRNEWGCKCNDSSD

SEQ ID NO: 23 - protein, 23 aa, Influenza virus
SLLTEVETPIRNEWGCRCNGSSD

SEQ ID NO: 24 - protein, 23 aa, Influenza virus
SLLTEVETPTRNEWGCRCNDSSD

SEQ ID NO: 25 - protein, 23 aa, Influenza virus
SLLTEVETPIRSEWGCRCNDSSD

SEQ ID NO: 26 - protein, 23 aa, Influenza virus
SLPTEVETPIRNEWGCRCNDSSD

SEQ ID NO: 27 - protein, 23 aa, Influenza virus
SLLTEVETPIRSEWGCRCNDSGD

SEQ ID NO: 28 - protein, 23 aa, Influenza virus
SLLTEVETPIRNEWECRCNDSSD

SEQ ID NO: 29 - protein, 23 aa, Influenza virus
SLLTEVETTISNEWGCRCNDSSD

SEQ ID NO: 30 - protein, 23 aa, Influenza virus
SLLTEVETHIRNEWDCRCNGSSD

SEQ ID NO: 31 - protein, 23 aa, Influenza virus
SLLTEVETPIRNEWECRCNGSSD

SEQ ID NO: 32 - protein, 23 aa, Influenza virus
SLLTEVETPIRNGWECKCNDSSD

SEQ ID NO: 33 - protein, 23 aa, Influenza virus
SLLTEVELPIRNEWGCRCNGSSD

SEQ ID NO: 34 - protein, 23 aa, Influenza virus
SLLTEVETPTRNGWGCRCSDSSD

SEQ ID NO: 35 - protein, 23 aa, Influenza virus
SLLTEVETPTRNGWGCRFSDSSD

SEQ ID NO: 36 - protein, 23 aa, Influenza virus
SLLTEVETPTRNGWGCRYSDSSD

SEQ ID NO: 37 - protein, 23 aa, Influenza virus
SLLTEVETPIRNEWGCKCNDSSD

SEQ ID NO: 38 - protein, 23 aa, Influenza virus
SLLTEVETPIRNEWEYRCSDSSD

SEQ ID NO: 39 - protein, 46 aa, Influenza virus
SLLTEVETPIRNEWGCRCNDSSNSFLPEVETPIRNEWGCRCNDSSD

SEQ ID NO: 40 - protein, 23 aa, Influenza virus
SLLTEVETPIRNEWGCRCNDSND

SEQ ID NO: 41 - protein, 23 aa, Influenza virus
SFLTEVETPIRNEWGCRCNDSSD

SEQ ID NO: 42 - protein, 23 aa, Influenza virus
SLLTEVETPIKNEWGCRCNDSSD

SEQ ID NO: 43 - protein, 22 aa, Influenza virus
LLTEVETPIRNEWGCRCNDSSD

SEQ ID NO: 44 - protein, 23 aa, Influenza virus
SLLPEVETPIRNEWGCRCNDSSD

SEQ ID NO: 45 - protein, 23 aa, Influenza virus
SLLTEVETPIRNEWGCRSNDSSD

SEQ ID NO: 46 - protein, 23 aa, Influenza virus
SLLTEVETPIRKEWGCRCNDSSD

SEQ ID NO: 47 - protein, 23 aa, Influenza virus
SLLTEVETPIKNEWGCRCNDSND

SEQ ID NO: 48 - protein, 21 aa, Influenza virus
LTEVETPIRNEWGCRCNDSSD

SEQ ID NO: 49 - protein, 22 aa, Influenza virus
LLTEVETPIRDEWGCRCNDSSD

SEQ ID NO: 50 - protein, 23 aa, Influenza virus
SLPTEVETPIRSEWGCRCNDSSD

SEQ ID NO: 51 - protein, 21 aa, Influenza virus
LTEVETPFRNEWGCRCNDSSD

SEQ ID NO: 52 - protein, 23 aa, Influenza virus
SLPTEVETPIRSEWGCRCNDSSD

SEQ ID NO: 53 - protein, 21 aa, Influenza virus
LTEVETPIRNEWGCRCNDSND

SEQ ID NO: 54 - protein, 20 aa, Influenza virus
TEVETPIRNEWGCRCNDSSD

SEQ ID NO: 55 - protein, 21 aa, Influenza virus
LTEVETPTRNEWGCRCNDSSD

SEQ ID NO: 56 - protein, 21 aa, Influenza virus
LTEVETPTKNEWGCRCNDSSD

SEQ ID NO: 57 - protein, 23 aa, Influenza virus
SLLTEVETPIRSGWECKCNDSSD

SEQ ID NO: 58 - protein, 23 aa, Influenza virus
SLLTEVETPTRNGWECRCSDSSD

SEQ ID NO: 59 - protein, 23 aa, Influenza virus
SLLTEVETPTRNGWECRCNDSSD

SEQ ID NO: 60 - protein, 23 aa, Influenza virus
SLLTEVETPTRNEWECRCSDSSD

SEQ ID NO: 61 - protein, 23 aa, Influenza virus
SLLTEVETPTRNEWECKCSDSSD

SEQ ID NO: 62 - protein, 22 aa, Influenza virus
LLTEVETPTRNEWECRCSDSSD

SEQ ID NO: 63 - protein, 23 aa, Influenza virus
SLLTEVETPTRNEWECRCSGSSD

SEQ ID NO: 64 - protein, 23 aa, Influenza virus
SLLTEVETLTRNGWGCRCSDSSD

SEQ ID NO: 65 - protein, 23 aa, Influenza virus
SLLTEVETPTKNEWECKCSDSSD

SEQ ID NO: 66 - protein, 23 aa, Influenza virus
SLLTEVETPTRNEWECRYSDSSD

SEQ ID NO: 67 - protein, 23 aa, Influenza virus
SLLTEVETLTRNEWECRCSDSSD

SEQ ID NO: 68 - protein, 23 aa, Influenza virus
SLLTEVETPTRNEWECKCSGSSD

SEQ ID NO: 69 - protein, 23 aa, Influenza virus
SLLTEVETLTKNGWGCRCSDSSD

SEQ ID NO: 70 - protein, 23 aa, Influenza virus
SLLTEVETPTRNDWECKCSDSSD

SEQ ID NO: 71 - protein, 23 aa, Influenza virus
SLLTEVETPIKSGWECRCNDSSD

SEQ ID NO: 72 - protein, 23 aa, Influenza virus
SLLTEVETPTRNGWGCRCSGSSD

SEQ ID NO: 73 - protein, 23 aa, Influenza virus
SLLTEVETPTRNGWECKCNDSSD

SEQ ID NO: 74 - protein, 23 aa, Influenza virus
SLLTEVETHTRNGWGCRCSDSSD

SEQ ID NO: 75 - protein, 23 aa, Influenza virus
SLLPEVETPTRNGWGCRCSGSSD

SEQ ID NO: 76 - protein, 23 aa, Influenza virus
SLLTEVETPIRNEWECRCSDSSD

SEQ ID NO: 77 - protein, 22 aa, Influenza virus
LLTEVETPIRNEWECRCSDSSD

SEQ ID NO: 78 - protein, 498 aa, Influenza virus
MASQGTKRSYEQMETDGDRQNATEIRASVGKMIDGIGRFYIQMCTELKLSDYEGRLIQNSLTIEKMVLSAFDERRNRYLEEHPSAGKDPKKTGGPIYRRVDGKWMRELVLYDKEEIRRIWRQANNGEDATAGLTHMMIWHSNLNDATYQRTRALVRTGMDPRMCSLMQGSTLPRRSGAAGAAVKGIGTMVMELIRMYKRGNGRKTRSAYERMCNILKGKFQTAAQRAMVDINDRNFWRGEQVRESRNPGNAEIEDLIFLARSALILRGSVAHKSCLPACVYGPAVSSGYDFEKEGYSLVGIDPFKLLQNSQVYSLIRPNENPAHKSQLVWMACHSAAFEDLRLLSFIRGTKVSPRGKLSTRGVQIASNENMDNMGSSTLELRSGYWAIRTRSGGNTNQQRASAGQISVQPTFSVQRNLPFEYSTVMAAFTGNTEGRTSDMRAEIIRMMEGAKPEEVSFRGRGVFELSDEKATNPIVPSFDMSNEGSYFFGDNAEEYDN

SEQ ID NO: 79 - protein, 498 aa, Artificial Sequence
  Syntethic construct
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGNGRKTRIAYERMCNILKGKFQTAAQKAMMDINDRNFWRGEQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRIEIIRMMESARPEDVSFRGQGVFELSDEKAASPIVPSFDMSNEGSYFFGDNAEEYDN